(19) United States
(12) Patent Application Publication
(10) Pub. No.: US 2009/0118276 A1
(43) Pub. Date: May 7, 2009

(54) THIENOPYRIMIDINES, THIENOPYRIDINES, AND PYRROLOPYRIMIDINES AS B-RAF INHIBITORS

(75) Inventors: Ariamala Gopalsamy, Mahwah, NJ (US); Mengxiao Shi, Eastchester, NY (US); Dan Maarten Berger, New City, NY (US); Minu Dhanjisha Dutia, West Nyack, NY (US); Nancy Torres, Nyack, NY (US); Darrin William Hopper, New York, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/262,622

(22) Filed: Oct. 31, 2008

Related U.S. Application Data
(60) Provisional application No. 60/984,918, filed on Nov. 2, 2007.

(51) Int. Cl. A61K 31/5377; C07D 495/04; C07D 487/04; A61K 31/519

(52) U.S. Cl. 514/234.2; 544/278; 514/260.1; 544/117; 544/280; 514/265.1

(57) ABSTRACT

The present invention relates to compounds of formula la:

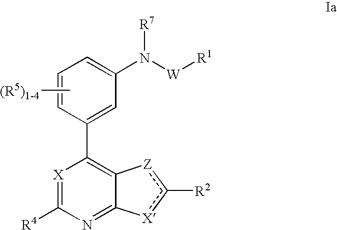

and pharmaceutically acceptable salts thereof. The thieno[3,2-d]pyrimidine, thieno[2,3-d]pyrimidine, thieno[3,2-b]pyridine, thieno[2,3-b]pyridine, and pyrrolo[2,3-d]pyrimidine compounds selectively inhibit B-Raf kinase activity and are useful for treating disorders mediated by B-Raf kinase, and for the treatment of cancer.

### CROSS-REFERENCE TO RELATED APPLICATIONS

This application claims the benefit under 35 U.S.C. §119(e) to co-pending U.S. Provisional Application Ser. No. 60/984,918, filed Nov. 2, 2007, which is hereby incorporated by reference in its entirety.

### FIELD OF THE INVENTION

The present invention relates to new heterocyclic compositions that are useful for inhibiting abnormal growth of certain cell types. The invention is directed to certain substituted thieno[3,2-d]pyrimidines, thieno[2,3-d]pyrimidines, thieno[3,2-b]pyridines, thieno[2,3-b]pyridines, and pyrrolo[2,3-d]pyrimidines, their corresponding pharmaceutically acceptable salts, and methods for their preparation and use. The substituted thienopyrmidines, thienopyridines, and pyrrolopyrmidines inhibit growth of tumor cells, which contain oncogenic forms of Receptor Tyrosine Kinases, K-Ras and B-Raf kinase.

### BACKGROUND OF THE INVENTION

B-Raf kinase is one of three known Raf oncoprotein kinases involved in transmission of mitogenic and anti-apoptotic signals. B-Raf encodes a Ras-regulated kinase that mediates cell growth and malignant transformation pathway activation that controls cell growth and survival. Activation of a Ras/Raf/MEK pathway results in a cascade of events from the cell surface to the cell nucleus, ultimately affecting cell proliferation, apoptosis, differentiation and transformation. Activating B-Raf mutations have been found in 66% of malignant melanomas and in a smaller fraction of other cancers including those of the colorectum, as reported by Davies H., et al. (2002) Nature 417:906 and by Rajagopalan H., et al. (2002) Nature 418:934. Recently, B-raf has been shown to be frequently mutated in various human cancers, as described by Wan et al. (2004) Cell 116:855-867. Therefore, it is desirable to identify and characterize compounds that inhibit growth of tumor cells, which contain oncogenic forms of Receptor Tyrosine Kinases, K-Ras and B-Raf kinase.

Certain thienopyrimidines, thienopyridines, and pyrrolopyrimidines have been noted as kinase inhibitors. For example, U.S. Pat. Appl. Publ. No. 2007/0082880 A1 describes a group of thieno[2,3-b]pyridine-5-carbonitriles as protein kinase inhibitors which preferably have heteroaryls or bicyclic aryls such as indolyl or benzimidazole substituted on the pyridine ring. Those compounds differ in the placement and nature of the substituents relative to the compounds disclosed herein.

Variably-substituted thieno[3,2-d]pyrimidine, thieno[2,3-d]pyrimidine, thieno[3,2-b]pyridine, thieno[2,3-b]pyridine, and pyrrolo[2,3-d]pyrimidine compositions in accordance with embodiments of the present invention selectively inhibit B-Raf kinase activity and are useful for treating disorders mediated by B-Raf kinase and for the treatment of cancer including, for example, but not limited to, colonic polyps, in mammals.

### SUMMARY OF THE INVENTION

Accordingly, the invention provides in one embodiment, a compound of formula Ia:

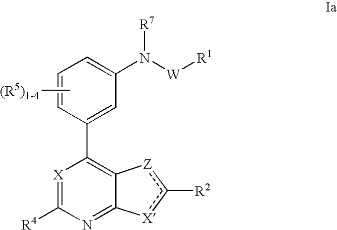

and pharmaceutically acceptable salts thereof;
wherein R¹ is selected from phenyl, heterocyclic ring and heteroaryl ring containing 1 to 3 ring heteroatoms selected from nitrogen, oxygen, and sulfur; wherein said phenyl, heterocyclic, and heteroaryl ring are each optionally substituted with from one to four substituents independently selected from the group consisting of -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, and YR¹⁰;
R² and R³ are independently selected from —H, -J, —C(O)OR⁷, —C(O)NR⁷R⁷, —NR⁶C(O)R⁷, —CN, heterocyclic ring and heteroaryl ring having 5 to 7 ring atoms and containing 1 to 3 ring heteroatoms selected from nitrogen, oxygen, and sulfur, and C₆-C₁₄ aryl ring, wherein the —R⁷ group, and said heterocyclic, heteroaryl and aryl rings are each optionally substituted with from one to four substituents independently selected from the group consisting of -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, and —YR¹⁰;
R⁴ is selected from —H, C₁-C₈ alkyl, C₂-C₆ alkenyl, and C₂-C₆ alkynyl;
R⁵, at each occurrence, is from one to four substituents independently selected from the group consisting of —H, -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)N R⁷R⁷, and YR¹⁰;
R⁶, at each occurrence, is selected from —H, —C(O)OR⁷, —C(O)NR⁷R⁷, C₃-C₁₀ carbocyclic ring, heterocyclic ring and heteroaryl ring having 5 to 7 ring atoms and containing 1 to 3 ring heteroatoms selected from nitrogen, oxygen, and sulfur, and C₆-C₁₄ aryl ring, wherein the R⁷ groups, carbocyclic, heterocyclic, heteroaryl and aryl rings are optionally substituted with from one to four substituents independently selected from the group consisting of -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, ——C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, and —R¹⁰;
R⁷, at each occurrence, is selected from —H, C₁-C₈ alkyl, C₂-C₆ alkenyl, and C₂-C₆ alkynyl;
R⁸ is a divalent group selected from C₁-C₆ alkyl, C₂-C₆ alkenyl, and C₂-C₆ alkynyl;
R⁹ is a divalent C₂-C₆ alkyl group;
R¹⁰, at each occurrence, is selected from the group consisting of: a C₃-C₁₀ carbocyclic ring; a heterocyclic ring and a heteroaryl ring containing 1 to 3 ring heteroatoms selected from nitrogen, oxygen, and sulfur; and a C₆-C₁₄ aryl ring; wherein the aryl, carbocyclic, heterocyclic and heteroaryl rings are optionally substituted with from one to four substituents independently selected from the group consisting of —H, -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷—, —OC(O)OR⁷, —OC(O)NR⁷R⁷, —NR⁷C(O)R⁷, —NR⁷C(O)OR⁷, —NR⁷C(O)NR⁷R⁷, —R⁸OR⁷, R⁸NR⁷R⁷, —R⁸S(O)ₘR⁷, —R⁸C(O)R⁷, —R⁸C(O)OR⁷, —R⁸C(O)NR⁷R⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, and —R⁸NR⁷C(O)NR⁷R⁷;
J is selected from fluoro, chloro, bromo, and iodo;
m is an integer from 0 to 2;
W is —C(O)— or —C(O)NR⁷—;
X is N, C—CN or C—C(O)NH₂;
X′ is —S—, —N(R⁶)—, or ═C(R³)—;
Z is —S— or ═C(R³)—, with the proviso wherein only one of X′ and Z is ═C(R³)—;
Y is selected from a bond, —NH—, —O—, —NR⁷—, and R⁸; and
[symbol] represents a single bond or a double bond.

An embodiment of the present invention provides a method for making a compound of formula Ia and pharmaceutically acceptable salts thereof:

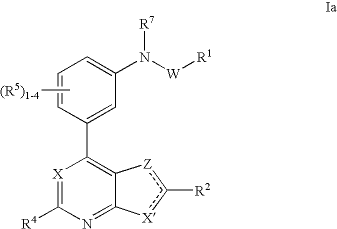

comprising the steps of reacting, in the presence of a palladium catalyst, a heterocyclic compound of formula II

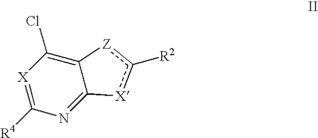

with a boronic acid of formula III,

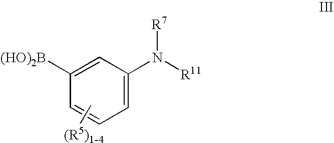

to obtain a compound of formula If,

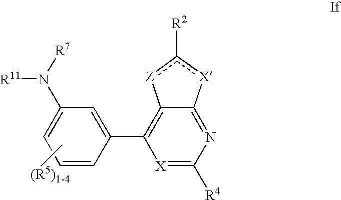

and, when R¹¹ is H, further reacting the compound obtained with an electrophile containing the —W—R¹ radical,
wherein W, X, X′, Z, R¹, R², R⁴, R⁵ and R⁷ are as defined above, and R¹¹ is —H or —W—R¹.

The invention also provides methods for inhibiting B-Raf kinase activity in a cell comprising contacting a cell with compounds of formula A, whereby the compound inhibits B-raf kinase activity.

The present invention also provides a method of treating a B-Raf kinase-dependent condition, especially inflammation or cancer, by administering to a patient a compound of formula A.

The present invention provides methods of treating mammalian diseases associated with B-Raf kinase by administering to a patient a compound of formula A.

The present invention provides methods of treating cancer selected from the group consisting of: breast, kidney, bladder, mouth, larynx, esophagus, stomach, colon, ovary, lung, pancreas, skin, liver, prostate and brain cancer.

### DETAILED DESCRIPTION OF THE INVENTION

Unless otherwise indicated, the following terms are held to have the following meanings as used herein.

The term “alkyl” means a straight and branched hydrocarbon chain containing from 1 to 8 carbon atoms.

The term “alkenyl” means a branched or straight chain having from 2 to 6 carbon atoms, the chain containing at least one carbon-carbon double bond which may exist in the E or Z configuration; the compounds of this invention include both configurations.

The term “alkynyl” means a branched or straight chain having from 2 to 6 carbon atoms that contains at least one carbon-carbon triple bond.

The term “carbocycle” means a non-aromatic, saturated or unsaturated hydrocarbon ring having 3 to 10 carbon atoms as a monocycle, bridged bi- and tricyclic rings, or spirocyclic rings.

The term “aryl” means a aromatic hydrocarbon moiety of 6 to 14 carbon atoms and which may be monocyclic, or multiple rings fused together, wherein at least one of the rings is aromatic. The aryl moiety may be substituted at any suitable ring position as provided herein. In addition to other optional substituents, the aryl group may be substituted by an oxo substituent meaning one of the ring carbon atoms is part of a carbonyl group.

The term “heteroaryl” means an aromatic heterocyclic ring system having from 5-14 ring atoms, which may be a single ring or multiple rings fused together or linked covalently, wherein at least one of the rings is aromatic. The rings contain from 1 to 4 heteroatoms independently selected from nitrogen, oxygen, or sulfur, wherein the nitrogen or sulfur atom(s) are optionally oxidized, or the nitrogen atom(s) are optionally quaternized. Any suitable ring position of the heteroaryl moiety may be covalently linked to the defined chemical structure; for example, the heteroaryl may be C-attached or N-attached where such is possible. The heteroaryl group may be substituted at any suitable ring position as provided herein. In addition to other optional substituents, the heteroaryl group may be substituted by an oxo substituent meaning one of the ring carbon atoms is part of a carbonyl group.

The term “heterocycle”, “heterocyclic” or “heterocyclyl” as used herein means a stable, saturated or partially unsaturated (i.e., having one or more double and/or triple bonds within the ring) monocyclic or multicyclic heterocyclic ring system having from 3 to 14 ring atoms, in which from 1 to 4 ring atoms are heteroatoms independently selected from nitrogen, oxygen, and sulfur, and the remaining ring atoms are carbon; the nitrogen or sulfur atom(s) are optionally oxidized, or the nitrogen atom(s) are optionally quaternized. The heterocyclic, heterocycle or heterocyclyl group may be substituted at any suitable ring position as provided herein. The heterocycle may be fused with a carbocyclic, heterocyclic, aryl or heteroaryl ring. Any suitable ring position of the heterocyclic moiety may be covalently linked to the defined chemical structure; e.g., the heterocycle may be C-attached or N-attached where such is possible. In addition to other optional substituents, the heterocyclic, heterocycle or heterocyclyl group may be substituted by an oxo substituent meaning one of the ring carbon atoms is part of a carbonyl group.

Unless expressly stated otherwise, any alkyl, alkenyl, alkynyl, aryl, heterocyclic, heteroaryl, or carbocyclic group may be optionally substituted with one or more groups selected from, but not limited to, alkyl, carbocycyl, heterocyclyl, halogen, haloalkyl, hydroxyalkyl, nitro, amino, hydroxyl, cyano, alkylamino, dialkylamino, alkoxyl, haloalkoxy, alkoxyalkyl, alkoxyalkoxy, oxo, aryl, aryloxy, heteroaryl, heteroaryloxy, acyl, carboxyl, sulfonyl, carboalkoxy, carbamoyl, alkylcarbamoyl, alkanoyloxy, and, sulfonyl, sulfonamido

As used herein, the term “pharmaceutically acceptable carrier” includes pharmaceutically acceptable diluents and excipients.

As used herein, the term “individual”, “subject” or “patient,” used interchangeably, refers to any animal, including mammals, preferably mice, rats, other rodents, rabbits, dogs, cats, swine, cattle, sheep, horses, or primates, and most preferably humans.

An embodiment of the present invention provides compounds of formula Ia and pharmaceutically acceptable salts thereof,

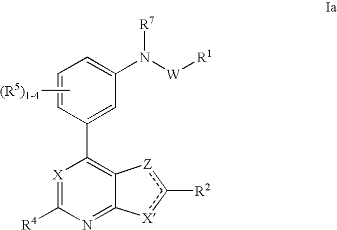

wherein R¹ is selected from phenyl, heterocyclic ring and heteroaryl ring containing 1 to 3 ring heteroatoms selected from nitrogen, oxygen, and sulfur; wherein phenyl, heterocyclic, and heteroaryl ring are optionally substituted with from one to four substituents independently selected from the group consisting of -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, and YR¹⁰;
R² and R³ are independently selected from —H, -J, —C(O)OR⁷, —C(O)NR⁷R⁷, —NR⁶C(O)R⁷, —CN, heterocyclic ring and heteroaryl ring having 5 to 7 ring atoms and containing 1 to 3 ring heteroatoms selected from nitrogen, oxygen, and sulfur, and C₆-C₁₄ aryl ring, wherein —R⁷, heterocyclic, heteroaryl and aryl rings are optionally substituted with from one to four substituents independently selected from the group consisting of -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)O R⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, and —YR¹⁰;
R⁴ is selected from —H, C₁-C₈ alkyl, C₂-C₆ alkenyl, and C₂-C₆ alkynyl;
R⁵ is from one to four substituents independently selected from the group consisting of —H, -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, and YR¹⁰;
R⁶ is selected from —H, —C(O)OR⁷, —C(O)NR⁷R⁷, C₁-C₈ alkyl, C₃-C₁₀ carbocyclic ring, heterocyclic ring and heteroaryl ring having 5 to 7 ring atoms and containing 1 to 3 ring heteroatoms selected from nitrogen, oxygen, and sulfur, and C₆-C₁₄ aryl ring, wherein the R⁷ and alkyl groups, and the carbocyclic, heterocyclic, heteroaryl and aryl rings are optionally substituted with from one to four substituents independently selected from the group consisting of -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, and —R¹⁰;
R⁷ is selected from —H, C₁-C₈ alkyl, C₂-C₆ alkenyl, and C₂-C₆ alkynyl;
R⁸ is a divalent group selected from C₁-C₆ alkyl, C₂-C₆ alkenyl, and C₂-C₆ alkynyl;
R⁹ is a divalent C₂-C₆ alkyl group;
R¹⁰ is a C₃-C₁₀ carbocyclic ring; heterocyclic ring and heteroaryl ring containing 1 to 3 ring heteroatoms selected from nitrogen, oxygen, and sulfur, and C₆-C₁₄ aryl ring; wherein the aryl, carbocyclic, heterocyclic and heteroaryl rings are optionally substituted with from one to four substituents independently selected from the group consisting of —H, -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷—, —OC(O)OR⁷, —OC(O)NR⁷R⁷, —NR⁷C(O)R⁷, —NR⁷C(O)OR⁷, —NR⁷C(O)NR⁷R⁷, —R⁸OR⁷, R⁸NR⁷R⁷, —R⁸S(O)ₘR⁷, —R⁸C(O)R⁷, —R⁸C(O)OR⁷, —R⁸C(O)NR⁷R⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, and —R⁸NR⁷C(O)NR⁷R⁷;
J is selected from fluoro, chloro, bromo, and iodo;
m is an integer from 0 to 2;
W is —C(O)— or —C(O)NR⁷—;
X is N, C—CN or C—C(O)NH₂;
X′ is —S—, —N(R⁶)—, or ═C(R³)—;
Z is —S— or ═C(R³)—, with the proviso wherein only one of X′ and Z is ═C(R³)—;
Y is selected from a bond, —NH—, —O—, —NR⁷—, and R⁸; and
[symbol] represents a single bond or a double bond.

In one embodiment of the present invention, thieno[2,3-d]pyrimidines, and thieno[2,3-b]pyridine compounds of formula Ib, and pharmaceutically acceptable salts thereof, wherein W, X, R¹, R², R³, R⁴, R⁵and R⁷ are as defined herein, are provided.

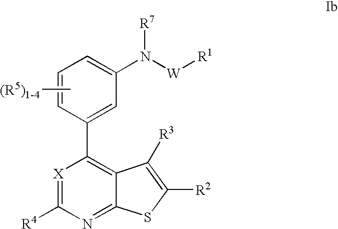

In another embodiment of the present invention, thieno[3,2-d]pyrimidines and thieno[3,2-b]pyridines compounds of formula Ic, and pharmaceutically acceptable salts thereof, wherein W, X, R¹, R², R³, R⁴, R⁵and R⁷are as defined herein, are provided.

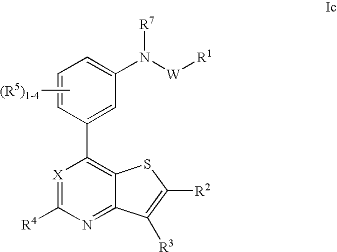

Another embodiment of the present invention provides pyrrolo[2,3-d]pyrimidines and pharmaceutically acceptable salts thereof, of formula Id, wherein W, X, R¹, R², R³, R⁴, R⁵, R⁶, and R⁷ are as defined herein.

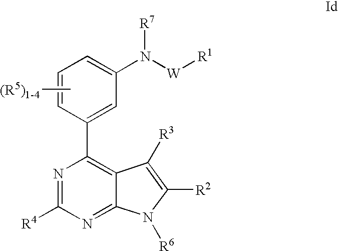

In accordance with embodiments of the invention, nonlimiting examples of R¹ include phenyl, pyridinyl, indanyl, indolyl, morpholinyl, piperidinyl, and piperazinyl. In one embodiment, R¹ is selected from the group consisting of 3-trifluoromethylphenyl, 4-fluoro-3-trifluoromethylphenyl, 4-methyl-3-trifluoromethylphenyl, 4-chloro-3-trifluoromethylphenyl, 4-methoxy-3-trifluoromethylphenyl, 3,4-dimethylphenyl 4-methylphenyl, 3-methylphenyl, 2-methylphenyl, 4-bromophenyl, 3-bromophenyl, 2-bromophenyl, 4-fluorophenyl, 3-fluorophenyl, 2-fluorophenyl, 4-chlorophenyl, 3-chlorophenyl, 2-chlorophenyl and 3,4-dichlorophenyl.

According to embodiments of the invention, nonlimiting examples of R² include phenyl, piperidin-4-yl, 1,2,3,6-tetrahydropyridin-4-yl, 4-pyridinyl, 3-pyridinyl, and 2-pyridinyl, wherein the foregoing are optionally substituted with from one to four substituents independently selected from the group consisting of -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, and —R¹⁰. In one embodiment, R² is selected from the group consisting of 4-methylphenyl, 3-methylphenyl, 2-methylphenyl, 4-aminophenyl, 4-acetamidophenyl, 4-chlorophenyl, 3-chlorophenyl, 2-chlorophenyl, 4-hydroxypiperidin-4-yl, N-methyl-5-picolinamido, N,N-dimethyl-4-carbamoylphenyl, N,N-dimethyl-3-carbamoylphenyl, 4-acetylphenyl, and 3-acetylphenyl.

In accordance with embodiments of the invention, nonlimiting examples of R⁶ include C₁-C₈ alkyl, amino-substituted C₁-C₈ alkyl, aryl-substituted C₁-C₈ alkyl, heterocycyl-substituted C₁-C₈ alkyl, sulfonyls, cyano-substituted phenyl, carboxyl-substituted phenyl, alkyl-substituted phenyl, alkoxy-substituted phenyl, amino-substituted phenyl, heterocycyl-substituted phenyl, pyrrolidinyl, piperidinyl, piperazinyl, morpholinyl, N-acetylated heterocycles, pyridinyl, and substituted pyridinyl. In one embodiment, R³ is selected from the group consisting of methylsulfonyl, phenylsulfonyl, tosyl, pyridin-4-yl, 4-(piperazin-1-yl-methyl)phenyl, 2-aminopyrimidin-5-yl, 4-(dimethylamino)methylphenyl, benzyl, 3-dimethylaminopropyl, and 2-(pyrrolidin-1-yl)ethyl.

The compounds of this invention may be prepared from (a) commercially available starting materials, (b) known starting materials that may be prepared as described in literature procedures, or (c) new intermediates described in the schemes and experimental procedures herein.

Reactions are performed in a solvent appropriate to the reagents and materials employed and suitable for the transformation being effected. It is understood by those skilled in the art of organic synthesis that the various functionalities present on the molecule must be consistent with the chemical transformation proposed. This may necessitate routine judgment as to the order of synthetic steps, and the need for protecting groups for remote functionalities.

Compounds of the present invention may be prepared as illustrated in the examples and in the following reaction schemes.

Referring now to Scheme 1, compounds of formula Ia can be obtained via coupling of compound II with boronic acid III, wherein R¹¹ is —H or —W—R¹, in the presence of a palladium catalyst, for example tetrakistriphenylphospinepalladium, in a mixture of DME and aqueous sodium bicarbonate.

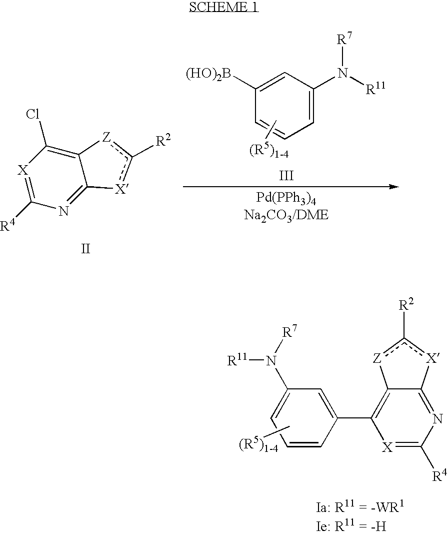

When R¹¹ is H, the resulting coupling product Ie can react with electrophiles, including, but not limited to, acid chlorides, isocyanates, anhydrides, and activated anhydrides and esters such as 1-hydroxybenzotriazole esters and mixed carboxylic phosphoric acid anhydrides generated by the reaction of a carboxylic acid with an activating agent such as 1-hydroxybenzotriazole (HOBT), diphenylphosphoryl azide (DPPA), and similar agents known by those skilled in the art, to introduce —W—R¹ and obtain compounds of formula Ia.

The compound II may be available from commercial sources or known literature procedures, or may be prepared as illustrated in the following reaction schemes 2 to 4.

In reference to Scheme 2, compound IIa can be prepared from chloro-substituted thienopyrimidine and thienopyridine derivatives IV wherein one of X′ and Z is —S— and the other of X′ and Z is ═C(R³)— using methods analogous to those described in U.S. Pat. Appl. Publ. No. 2007/0082880 A1. Chloro-substituted thieno derivatives IV are treated with lithium diisopropylamide in an inert solvent with cooling, preferably to about —78° C., followed by addition of iodine to obtain intermediate V. Intermediate V undergoes a coupling reaction with a boronic acid of formula R²—B(OH)₂ in the presence of a palladium catalyst, for example tetrakis-triphenylphospinepalladium, in a mixture of DME and aqueous sodium bicarbonate to yield intermediate IIa. Alternatively, CO₂ can be used in place of I₂, and the coupling step omitted to obtain carboxylate derivatives of II, wherein R² is —C(O)OR⁷ or —C(O)NR⁷R⁷.

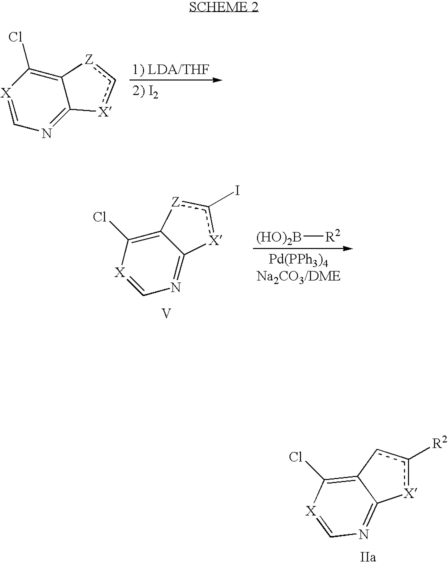

Chloro-substituted thienopyrimidine and thienopyridine derivatives IV wherein one of X′ and Z is —S— and the other of X′ and Z is ═C(R³)—, such as 4-chloro-thieno[3,2-d]pyrimidine, 4-chloro-thieno[2,3-d]pyrimidine, 4-chlorothieno[2,3-b]pyridine-5-carbonitrile, and 7-chlorothieno[3,2-b]pyridine-6-carboxamide, can be obtained from commercial sources or readily prepared by numerous literature procedures by those skilled in the art (using, for example, methods described in J. Med. Chem. 2004, 47, 6666-6668).

In an alternate method, in reference to schemes 3 and 4, when compound II is a 4-chloro-thieno[2,3-d]pyrimidine or a 4-chloro-pyrrolo[2,3-d]pyrimidine compound, compound I may be prepared by reacting amino acid derivatives of thiophene and pyrrole with substituted formamidines, and treating the product thus obtained with a chlorinating agent.

Referring now to Scheme 3,4-chloro-thieno[2,3-d]pyrimidine derivatives of formula IIb may be prepared by reaction of 2-amino-4-yl-thiophene-3-carboxylic acid ethyl ester VIa (prepared, for example, by methods analogous to the procedure described in PCT Appl. Publ. No. WO 2004/041813 A1) with an R⁴-substituted formamidine VII, preferably with heating to reflux in a suitable solvent, wherein R² and R⁴ are as defined herein. The thieno[2,3-d]pyrimidin-2-one VIIIa thus obtained is treated with a chlorinating agent such as thionyl chloride to yield 4-chloro-thieno[2,3-d]pyrimidine IIb.

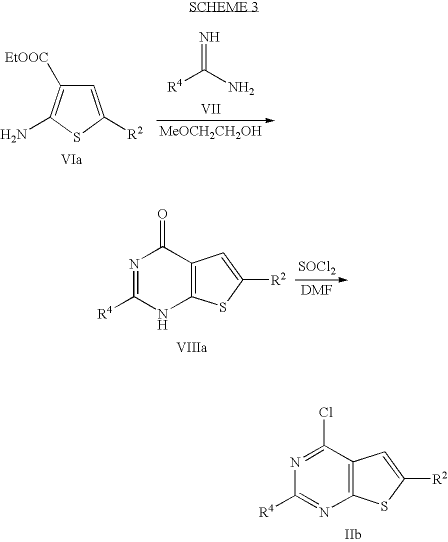

Referring now to Scheme 4,4-chloro-pyrrolo[2,3-d]pyrimidine derivatives of formula IIc may be prepared by reaction of ethyl 2-amino-5-pyridin-4-yl-1H-pyrrole-3-carboxylate VIb with an R⁴-substituted formamidine VII, preferably with heating to reflux in a suitable solvent, wherein R² and R⁴ are as defined herein. The pyrrolo[2,3-d]pyrimidin-2-one VIIIb thus obtained is treated with a chlorinating agent such as phosphorus oxychloride to yield 4-chloro-pyrrolo[2,3-d]pyrimidine IIc.

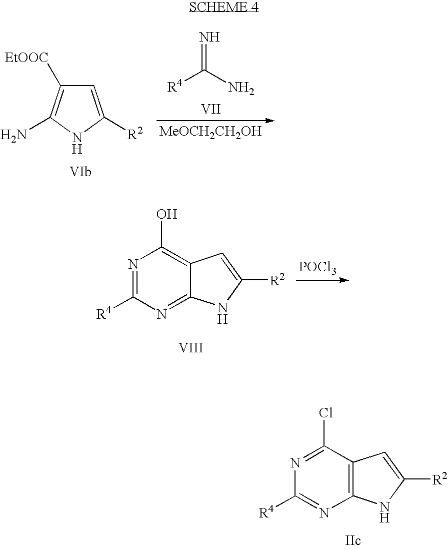

Nitriles may be used in place of R⁴-substituted formamidine VII in the synthetic pathways illustrated in Schemes 3 and 4. Further, compounds VIIIa and VIIIb may exist in a equilibrium of keto and enol forms as shown below; specific reference to any form herein encompasses all forms and should not be held to exclude the others:

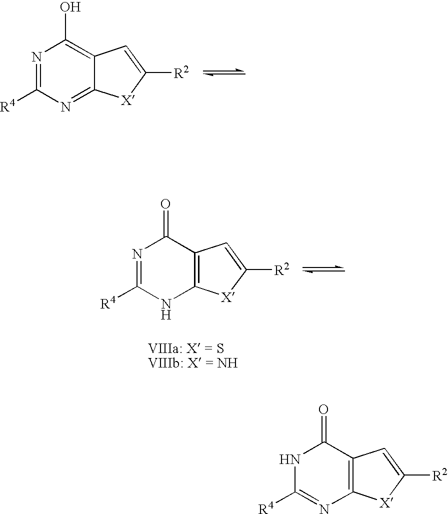

Exemplary compounds of formula Ia in accordance with embodiments of the present invention include the following compounds in Table 1:

TABLE 1

| Compound | |
|---|---|
| No. | Compound Name |
| 1 | N-(3-thieno[3,2-d]pyrimidin-4-yl-phenyl)-3-(trifluoromethyl)benzamide |
| 2 | N-(3-thieno[2,3-d]pyrimidin-4-yl-phenyl)-3-(trifluoromethyl)benzamide |
| 3 | N-[3-(7-methylthieno[3,2-d]pyrimidin-4-yl)phenyl]-3-(trifluoromethyl)benzamide |
| 4 | ethyl 4-(3-{[3-(trifluoromethyl)benzoyl]amino}phenyl)thieno[2,3-d]pyrimidine-6- |
| | carboxylate |
| 5 | N-[3-(6-cyano-2-pyridin-4-ylthieno[3,2-b]pyridin-7-yl)phenyl]-3- |
| | (trifluoromethyl)benzamide |
| 6 | N-[3-(6-pyridin-4-ylthieno[3,2-d]pyrimidin-4-yl)phenyl]-3-(trifluoromethyl)benzamide |
| 7 | N-[3-(6-cyano-2-pyridin-4-ylthieno[3,2-b]pyridin-7-yl)phenyl]-4-methoxy-3- |
| | (trifluoromethyl)benzamide |
| 8 | N-[3-(6-cyano-2-pyridin-4-ylthieno[3,2-b]pyridin-7-yl)phenyl]-N′-[4-fluoro-3- |
| | (trifluoromethyl)phenyl]urea |
| 9 | N-[3-(6-cyano-2-pyridin-4-ylthieno[3,2-b]pyridin-7-yl)phenyl]-4-methyl-3- |
| | (trifluoromethyl)benzamide |
| 10 | N-[3-(6-cyano-2-pyridin-4-ylthieno[3,2-b]pyridin-7-yl)phenyl]-4-fluoro-3- |
| | (trifluoromethyl)benzamide |
| 11 | N-[4-chloro-3-(trifluoromethyl)phenyl]-N′-[3-(6-cyano-2-pyridin-4-ylthieno[3,2-b]pyridin- |
| | 7-yl)phenyl]urea |
| 12 | 4-chloro-N-[3-(6-cyano-2-pyridin-4-ylthieno[3,2-b]pyridin-7-yl)phenyl]-3- |
| | (trifluoromethyl)benzamide |
| 13 | N-[3-(6-cyano-2-pyridin-4-ylthieno[3,2-b]pyridin-7-yl)phenyl]-N′-[3- |
| | (trifluoromethyl)phenyl]urea |
| 14 | 3,4-dichloro-N-[3-(6-cyano-2-pyridin-4-ylthieno[3,2-b]pyridin-7-yl)phenyl]benzamide |
| 15 | N-[3-(6-cyano-2-pyridin-4-ylthieno[3,2-b]pyridin-7-yl)phenyl]-N′-(3,4- |
| | dimethylphenyl)urea |
| 16 | N-(3-chlorophenyl)-N′-[3-(6-cyano-2-pyridin-4-ylthieno[3,2-b]pyridin-7-yl)phenyl]urea |
| 17 | N-(4-chlorophenyl)-N′-[3-(6-cyano-2-pyridin-4-ylthieno[3,2-b]pyridin-7-yl)phenyl]urea |
| 18 | 3-chloro-N-[3-(6-cyano-2-pyridin-4-ylthieno[3,2-b]pyridin-7-yl)phenyl]benzamide |
| 19 | N-[3-(6-cyano-2-pyridin-3-ylthieno[3,2-b]pyridin-7-yl)phenyl]-4-methoxy-3- |
| | (trifluoromethyl)benzamide |
| 20 | N-[3-(6-cyano-2-pyridin-3-ylthieno[3,2-b]pyridin-7-yl)phenyl]-N′-[4-fluoro-3- |
| | (trifluoromethyl)phenyl]urea |
| 21 | N-[3-(6-cyano-2-pyridin-3-ylthieno[3,2-b]pyridin-7-yl)phenyl]-4-methyl-3- |
| | (trifluoromethyl)benzamide |
| 22 | N-[3-(6-cyano-2-pyridin-3-ylthieno[3,2-b]pyridin-7-yl)phenyl]-4-fluoro-3- |
| | (trifluoromethyl)benzamide |
| 23 | N-[4-chloro-3-(trifluoromethyl)phenyl]-N′-[3-(6-cyano-2-pyridin-3-ylthieno[3,2-b]pyridin- |
| | 7-yl)phenyl]urea |
| 24 | 4-chloro-N-[3-(6-cyano-2-pyridin-3-ylthieno[3,2-b]pyridin-7-yl)phenyl]-3- |
| | (trifluoromethyl)benzamide |
| 25 | N-[3-(6-cyano-2-pyridin-3-ylthieno[3,2-b]pyridin-7-yl)phenyl]-N′-[3- |
| | (trifluoromethyl)phenyl]urea |
| 26 | N-[3-(6-cyano-2-pyridin-3-ylthieno[3,2-b]pyridin-7-yl)phenyl]-3- |
| | (trifluoromethyl)benzamide |
| 27 | 3,4-dichloro-N-[3-(6-cyano-2-pyridin-3-ylthieno[3,2-b]pyridin-7-yl)phenyl]benzamide |
| 28 | N-[3-(6-cyano-2-pyridin-3-ylthieno[3,2-b]pyridin-7-yl)phenyl]-N′-(3,4- |
| | dimethylphenyl)urea |
| 29 | N-(3-chlorophenyl)-N′-[3-(6-cyano-2-pyridin-3-ylthieno[3,2-b]pyridin-7-yl)phenyl]urea |
| 30 | N-(4-chlorophenyl)-N′-[3-(6-cyano-2-pyridin-3-ylthieno[3,2-b]pyridin-7-yl)phenyl]urea |
| 31 | 3-chloro-N-[3-(6-cyano-2-pyridin-3-ylthieno[3,2-b]pyridin-7-yl)phenyl]benzamide |
| 35 | 4-methoxy-N-[3-(6-pyridin-4-ylthieno[2,3-d]pyrimidin-4-yl)phenyl]-3- |
| | (trifluoromethyl)benzamide |
| 36 | N-[4-fluoro-3-(trifluoromethyl)phenyl]-N′-[3-(6-pyridin-4-ylthieno[2,3-d]pyrimidin-4- |
| | yl)phenyl]urea |
| 37 | 4-methyl-N-[3-(6-pyridin-4-ylthieno[2,3-d]pyrimidin-4-yl)phenyl]-3- |
| | (trifluoromethyl)benzamide |
| 38 | 4-fluoro-N-[3-(6-pyridin-4-ylthieno[2,3-d]pyrimidin-4-yl)phenyl]-3- |
| | (trifluoromethyl)benzamide |
| 39 | N-[4-chloro-3-(trifluoromethyl)phenyl]-N′-[3-(6-pyridin-4-ylthieno[2,3-d]pyrimidin-4- |
| | yl)phenyl]urea |
| 40 | 4-chloro-N-[3-(6-pyridin-4-ylthieno[2,3-d]pyrimidin-4-yl)phenyl]-3- |
| | (trifluoromethyl)benzamide |
| 41 | N-[3-(6-pyridin-4-ylthieno[2,3-d]pyrimidin-4-yl)phenyl]-N′-[3- |
| | (trifluoromethyl)phenyl]urea |
| 42 | N-[3-(6-pyridin-4-ylthieno[2,3-d]pyrimidin-4-yl)phenyl]-3-(trifluoromethyl)benzamide |
| 43 | 3,4-dichloro-N-[3-(6-pyridin-4-ylthieno[2,3-d]pyrimidin-4-yl)phenyl]benzamide |
| 44 | N-(3,4-dimethylphenyl)-N′-[3-(6-pyridin-4-ylthieno[2,3-d]pyrimidin-4-yl)phenyl]urea |
| 45 | N-(3-chlorophenyl)-N′-[3-(6-pyridin-4-ylthieno[2,3-d]pyrimidin-4-yl)phenyl]urea |
| 46 | N-(4-chlorophenyl)-N′-[3-(6-pyridin-4-ylthieno[2,3-d]pyrimidin-4-yl)phenyl]urea |
| 47 | 3-chloro-N-[3-(6-pyridin-4-ylthieno[2,3-d]pyrimidin-4-yl)phenyl]benzamide |
| 48 | 4-methoxy-N-[3-(5-pyridin-4-ylthieno[2,3-d]pyrimidin-4-yl)phenyl]-3- |
| | (trifluoromethyl)benzamide |
| 49 | N-[4-fluoro-3-(trifluoromethyl)phenyl]-N′-[3-(5-pyridin-4-ylthieno[2,3-d]pyrimidin-4- |
| | yl)phenyl]urea |
| 50 | 4-methyl-N-[3-(5-pyridin-4-ylthieno[2,3-d]pyrimidin-4-yl)phenyl]-3- |
| | (trifluoromethyl)benzamide |
| 51 | 4-fluoro-N-[3-(5-pyridin-4-ylthieno[2,3-d]pyrimidin-4-yl)phenyl]-3- |
| | (trifluoromethyl)benzamide |
| 52 | N-[4-chloro-3-(trifluoromethyl)phenyl]-N′-[3-(5-pyridin-4-ylthieno[2,3-d]pyrimidin-4- |
| | yl)phenyl]urea |
| 53 | 4-chloro-N-[3-(5-pyridin-4-ylthieno[2,3-d]pyrimidin-4-yl)phenyl]-3- |
| | (trifluoromethyl)benzamide |
| 54 | N-[3-(5-pyridin-4-ylthieno[2,3-d]pyrimidin-4-yl)phenyl]-N′-[3- |
| | (trifluoromethyl)phenyl]urea |
| 55 | N-[3-(5-pyridin-4-ylthieno[2,3-d]pyrimidin-4-yl)phenyl]-3-(trifluoromethyl)benzamide |
| 56 | 3,4-dichloro-N-[3-(5-pyridin-4-ylthieno[2,3-d]pyrimidin-4-yl)phenyl]benzamide |
| 57 | N-(3,4-dimethylphenyl)-N′-[3-(5-pyridin-4-ylthieno[2,3-d]pyrimidin-4-yl)phenyl]urea |
| 58 | N-(3-chlorophenyl)-N′-[3-(5-pyridin-4-ylthieno[2,3-d]pyrimidin-4-yl)phenyl]urea |
| 59 | N-(4-chlorophenyl)-N′-[3-(5-pyridin-4-ylthieno[2,3-d]pyrimidin-4-yl)phenyl]urea |
| 60 | 3-chloro-N-[3-(5-pyridin-3-ylthieno[2,3-d]pyrimidin-4-yl)phenyl]benzamide |
| 61 | N-[3-(5-cyano-2-pyridin-4-ylthieno[2,3-b]pyridin-4-yl)phenyl]-3- |
| | (trifluoromethyl)benzamide |
| 62 | N-[3-(5-cyano-2-pyridin-3-ylthieno[2,3-b]pyridin-4-yl)phenyl]-3- |
| | (trifluoromethyl)benzamide |
| 63 | N-[3-(6-pyridin-3-ylthieno[3,2-d]pyrimidin-4-yl)phenyl]-3-(trifluoromethyl)benzamide |
| 64 | tert-butyl 4-hydroxy-4-[4-(3-{[3-(trifluoromethyl)benzoyl]amino}phenyl)thieno[2,3- |
| | d]pyrimidin-6-yl]piperidine-1-carboxylate |
| 65 | N-{3-[6-(4-hydroxypiperidin-4-yl)thieno[2,3-d]pyrimidin-4-yl]phenyl}-3- |
| | (trifluoromethyl)benzamide |
| 66 | tert-butyl 4-[4-(3-{[3-(trifluoromethyl)benzoyl]amino}phenyl)thieno[2,3-d]pyrimidin-6-yl]- |
| | 3,6-dihydropyridine-1(2H)-carboxylate |
| 67 | N-{3-[6-(1,2,3,6-tetrahydropyridin-4-yl)thieno[2,3-d]pyrimidin-4-yl]phenyl}-3- |
| | (trifluoromethyl)benzamide |
| 68 | N-(3-{6-[4-(acetylamino)phenyl]thieno[2,3-d]pyrimidin-4-yl}phenyl)-3- |
| | (trifluoromethyl)benzamide |
| 69 | N-{3-[6-(4-aminophenyl)thieno[2,3-d]pyrimidin-4-yl]phenyl}-3- |
| | (trifluoromethyl)benzamide |
| 70 | N,N-dimethyl-4-[4-(3-{[3-(trifluoromethyl)benzoyl]amino}phenyl)thieno[2,3-d]pyrimidin- |
| | 6-yl]benzamide |
| 71 | N,N-dimethyl-3-[4-(3-{[3-(trifluoromethyl)benzoyl]amino}phenyl)thieno[2,3-d]pyrimidin- |
| | 6-yl]benzamide |
| 72 | N-(3-{6-[3-(acetylamino)phenyl]thieno[2,3-d]pyrimidin-4-yl}phenyl)-3- |
| | (trifluoromethyl)benzamide |
| 73 | N-(3-{6-[3-(dimethylamino)phenyl]thieno[2,3-d]pyrimidin-4-yl}phenyl)-3- |
| | (trifluoromethyl)benzamide |
| 74 | N-{3-[6-(3-aminophenyl)thieno[2,3-d]pyrimidin-4-yl]phenyl}-3- |
| | (trifluoromethyl)benzamide |
| 75 | N-[3-(6-pyridin-3-ylthieno[2,3-d]pyrimidin-4-yl)phenyl]-3-(trifluoromethyl)benzamide |
| 76 | N-{3-[6-(6-methoxypyridin-3-yl)thieno[2,3-d]pyrimidin-4-yl]phenyl}-3- |
| | (trifluoromethyl)benzamide |
| 77 | N-{3-[6-(4-cyanophenyl)thieno[2,3-d]pyrimidin-4-yl]phenyl}-3- |
| | (trifluoromethyl)benzamide |
| 78 | N-[3-(6-phenylthieno[2,3-d]pyrimidin-4-yl)phenyl]-3-(trifluoromethyl)benzamide |
| 79 | N-{3-[6-(4-chlorophenyl)thieno[2,3-d]pyrimidin-4-yl]phenyl}-3- |
| | (trifluoromethyl)benzamide |
| 80 | N-{3-[6-(4-acetylphenyl)thieno[2,3-d]pyrimidin-4-yl]phenyl}-3- |
| | (trifluoromethyl)benzamide |
| 81 | N-{3-[6-(3-acetylphenyl)thieno[2,3-d]pyrimidin-4-yl]phenyl}-3- |
| | (trifluoromethyl)benzamide |
| 82 | N-{3-[6-(4-methoxyphenyl)thieno[2,3-d]pyrimidin-4-yl]phenyl}-3- |
| | (trifluoromethyl)benzamide |
| 83 | N-{3-[6-(4-methylphenyl)thieno[2,3-d]pyrimidin-4-yl]phenyl}-3- |
| | (trifluoromethyl)benzamide |
| 84 | N-{3-[6-(3-methylphenyl)thieno[2,3-d]pyrimidin-4-yl]phenyl}-3- |
| | (trifluoromethyl)benzamide |
| 85 | N-methyl-4-[4-(3-{[3-(trifluoromethyl)benzoyl]amino}phenyl)thieno[2,3-d]pyrimidin-6- |
| | yl]pyridine-2-carboxamide |
| 86 | N-[3-(6-{2-[(2-morpholin-4-ylethyl)amino]pyridin-4-yl}thieno[2,3-d]pyrimidin-4- |
| | yl)phenyl]-3-(trifluoromethyl)benzamide |
| 87 | N-{3-[6-(2-{[3-(1H-imidazol-1-yl)propyl]amino}pyridin-4-yl)thieno[2,3-d]pyrimidin-4- |
| | yl]phenyl}-3-(trifluoromethyl)benzamide |
| 88 | N-[3-(6-cyanothieno[2,3-d]pyrimidin-4-yl)phenyl]-3-(trifluoromethyl)benzamide |
| 89 | 4-(3-{[3-(trifluoromethyl)benzoyl]amino}phenyl)thieno[2,3-d]pyrimidine-6-carboxamide |
| 90 | N-[3-(7H-pyrrolo[2,3-d]pyrimidin-4-yl)phenyl]-3-(trifluoromethyl)benzamide |
| 91 | N-(3-{7-[3-(dimethylamino)propyl]-7H-pyrrolo[2,3-d]pyrimidin-4-yl}phenyl)-3- |
| | (trifluoromethyl)benzamide |
| 92 | N-(3-{7-[(4-methylphenyl)sulfonyl]-7H-pyrrolo[2,3-d]pyrimidin-4-yl}phenyl)-3- |
| | (trifluoromethyl)benzamide |
| 93 | N-[3-(7-benzyl-7H-pyrrolo[2,3-d]pyrimidin-4-yl)phenyl]-3-(trifluoromethyl)benzamide |
| 94 | N-{3-[7-(2-cyanobenzyl)-7H-pyrrolo[2,3-d]pyrimidin-4-yl]phenyl}-3- |
| | (trifluoromethyl)benzamide |
| 95 | N-{3-[7-(3-cyanobenzyl)-7H-pyrrolo[2,3-d]pyrimidin-4-yl]phenyl}-3- |
| | (trifluoromethyl)benzamide |
| 96 | N-{3-[7-(4-cyanobenzyl)-7H-pyrrolo[2,3-d]pyrimidin-4-yl]phenyl}-3- |
| | (trifluoromethyl)benzamide |
| 97 | N-{3-[7-(pyridin-2-ylmethyl)-7H-pyrrolo[2,3-d]pyrimidin-4-yl]phenyl}-3- |
| | (trifluoromethyl)benzamide |
| 98 | N-{3-[7-(pyridin-4-ylmethyl)-7H-pyrrolo[2,3-d]pyrimidin-4-yl]phenyl}-3- |
| | (trifluoromethyl)benzamide |
| 99 | N-(3-{7-[4-(morpholin-4-ylmethyl)benzyl]-7H-pyrrolo[2,3-d]pyrimidin-4-yl}phenyl)-3- |
| | (trifluoromethyl)benzamide |
| 100 | N-(3-{7-[4-(2-methyl-1H-imidazol-1-yl)benzyl]-7H-pyrrolo[2,3-d]pyrimidin-4-yl}phenyl)- |
| | 3-(trifluoromethyl)benzamide |
| 101 | N-[3-(6-pyridin-4-yl-7H-pyrrolo[2,3-d]pyrimidin-4-yl)phenyl]-3- |
| | (trifluoromethyl)benzamide |
| 102 | N-{3-[7-(1-methylpiperidin-4-yl)-7H-pyrrolo[2,3-d]pyrimidin-4-yl]phenyl}-3- |
| | (trifluoromethyl)benzamide |
| 103 | N-{3-[7-(4-methoxyphenyl)-7H-pyrrolo[2,3-d]pyrimidin-4-yl]phenyl}-3- |
| | (trifluoromethyl)benzamide |
| 104 | N-{3-[7-(methylsulfonyl)-7H-pyrrolo[2,3-d]pyrimidin-4-yl]phenyl}-3- |
| | (trifluoromethyl)benzamide |
| 105 | N-{3-[7-(4-formylphenyl)-7H-pyrrolo[2,3-d]pyrimidin-4-yl]phenyl}-3- |
| | (trifluoromethyl)benzamide |
| 106 | N-{3-[7-(3-formylbenzyl)-7H-pyrrolo[2,3-d]pyrimidin-4-yl]phenyl}-3- |
| | (trifluoromethyl)benzamide |
| 107 | N-(3-{7-[4-(dimethylamino)phenyl]-7H-pyrrolo[2,3-d]pyrimidin-4-yl}phenyl)-3- |
| | (trifluoromethyl)benzamide |
| 108 | N-{3-[6-pyridin-4-yl-7-(2-pyrrolidin-1-ylethyl)-7H-pyrrolo[2,3-d]pyrimidin-4-yl]phenyl}-3- |
| | (trifluoromethyl)benzamide |
| 109 | N-(3-{7-[3-(dimethylamino)propyl]-6-pyridin-4-yl-7H-pyrrolo[2,3-d]pyrimidin-4- |
| | yl}phenyl)-3-(trifluoromethyl)benzamide |
| 110 | N-{3-[7-(2-aminopyrimidin-5-yl)-7H-pyrrolo[2,3-d]pyrimidin-4-yl]phenyl}-3- |
| | (trifluoromethyl)benzamide |
| 111 | N-[3-(7-{4-[(dimethylamino)methyl]phenyl}-7H-pyrrolo[2,3-d]pyrimidin-4-yl)phenyl]-3- |
| | (trifluoromethyl)benzamide |
| 112 | N-(3-{7-[4-(piperazin-1-ylmethyl)phenyl]-7H-pyrrolo[2,3-d]pyrimidin-4-yl}phenyl)-3- |
| | (trifluoromethyl)benzamide |

Pharmaceutically acceptable salts of the compounds of formula Ia with an acidic moiety may be formed from organic and inorganic bases. For example with alkali metals or alkaline earth metals such as sodium, potassium, lithium, calcium, or magnesium or organic bases and N-tetraalkylammonium salts such as N-tetrabutylammonium salts. Similarly, when a compound of this invention contains a basic moiety, salts may be formed from organic and inorganic acids. For example salts may be formed from acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids. Suitable examples of pharmaceutically acceptable salts include, but are not limited, to sulfate; citrate, acetate; oxalate; chloride; bromide; iodide; nitrate; bisulfate; phosphate; acid phosphate; isonicotinate; lactate; salicylate; acid citrate; tartrate; oleate; tannate; pantothenate; bitartrate; ascorbate; succinate; maleate; gentisinate; fumarate; gluconate; glucaronate; saccharate; formate; benzoate; glutamate; methanesulfonate; ethanesulfonate; benzenesulfonate; p-toluenesulfonate; pamoate (i.e., 1,1′-methylene-bis-(2-hydroxy-3-naphthoate)); and salts of fatty acids such as caproate, laurate, myristate, palmitate, stearate, oleate, linoleate, and linolenate salts. The compounds can also be used in the form of esters, carbamates and other conventional prodrug forms, which when administered in such form, convert to the active moiety in vivo.

The present invention accordingly provides a pharmaceutical composition, which comprises an effective amount of a compound of formula lain combination or association with a pharmaceutically acceptable carrier. Pharmaceutical compositions are prepared in accordance with acceptable pharmaceutical procedures, such as described in Remington's Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable. As used herein, the term “effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease; (2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and (3) ameliorating the disease; for example, ameliorating a disease, condition or disorder in an individual that is experiencing or displaying the pathology or symptomatology of the disease, condition or disorder (i.e., reversing the pathology and/or symptomatology).

### Standard Biological and Pharmacological Test Procedures

Evaluation of representative compounds of this invention in standard pharmacological test procedures indicated that the compounds of this invention possess significant anticancer activity and are in particular inhibitors of B-Raf kinase. Based on the activity shown in the standard pharmacological test procedures, the compounds of this invention are therefore useful as antineoplastic agents. In particular, these compounds are useful in treating, inhibiting the growth of, or eradicating neoplasms such as those of the breast, kidney, bladder, mouth, larynx, esophagus, stomach, colon, ovary, lung, pancreas, liver, prostate and skin.

### B-Raf Kinase to MEK1 ELISA

Representative examples of formula Ia were tested as B-Raf Kinase inhibitors that can inhibit growth of tumor cells that contain oncogenic forms of Receptor Tyrosine Kinases or K-Ras, or B-Raf kinase using the following Kinase Assay Protocol.

Reagents: Flag/GST-tagged recombinant human B-Raf produced in Sf9 insect cells, human non-active Mek-1-GST (recombinant protein produced in E. coli); and a phospho-MEK1 specific poly-clonal Ab from Cell Signaling Technology (Cat. #9121).

B-Raf1 Kinase Assay Procedure: B-Raf-1 is used to phosphorylate GST-MEK1. MEK1 phosphorylation is measured by a phospho-specific antibody (from Cell Signaling Technology, Cat. #9121) that detects phosphorylation of two serine residues at positions 217 and 221 on MEK1.

B-Raf Assay Stock Solutions:

1. Assay Dilution Buffer (ADB): 20 mM MOPS, pH 7.2, 25 mM β-glycerol phosphate, 5 mM EGTA, 1 mM sodium orthovanadate, 1 mM dithiothreitol, 0.01% Triton X-100.

2. Magnesium/ATP Cocktail: ADB solution (minus Triton X-100) plus 200 μM cold ATP and 40 mM magnesium chloride.

4. Active Kinase: Active B-Raf: used at 0.2 nM per assay point.

5. Non-active GST-MEK1: Use at 2.8 nM final concentration).

6. TBST-Tris (50 mM, pH 7.5), NaCl (150 mM), Tween-20 (0.05%)

7. Anti-GST Ab (GE)

8. Anti pMEK Ab (Upstate)

9. Anti-rabbit Ab/Europium conjugate (Wallac)

### Assay Procedure

1. Add 25 μl of ADB containing B-Raf and Mek per assay (i.e. per well of a 96 well plate)

2. Add 25 μl of 0.2 mM ATP and 40 mM magnesium chloride in Magnesium/ATP Cocktail.

3. Incubate for 45 minutes at RT with occasional shaking.

4. Transfer this mixture to an anti-GST Ab coated 96 well plate (Nunc Immunosorb plates coated o/n with a-GST. Plate freshly washed 3× with TBS-T before use.

5. Incubate o/n at 30° C. in cold room.

6. Wash 3× with TBST, add Anti-Phospho MEK1 (1:1000, dilution depends upon lot)

7. Incubate for 60 minutes at RT in a shaking incubator

8. Wash 3× with TBST, add Anti-rabbit Ab/Europium conjugate (Wallac) (1:500, dilution depends upon lot)

9. Incubate for 60 minutes at RT on a platform shaker.

10. Wash plate 3× with TBS-T

11. Add 100 μl of Wallac Delfia Enhancement Solution and shake for 10 minutes.

12. Read plates in Wallac Victor model Plate Reader.

13. Collect data analyze in Excel for single point and IC50 determinations. Mallon R, et al (2001) Anal. Biochem. 294:48.

### Analysis of Results

IC₅₀ determinations (Table 2) were performed on representative compounds of formula Ia from single point assays with >80% inhibition determined % inhibition at 10 mg/mL (% inhibition=1—sample treated with compound of Formula A/untreated control sample). The % inhibition was determined for each compound concentration. For IC₅₀ determinations, typically the B-Raf assay was run at compound concentrations from 1 μM to 3 nM or 0.1 μM to 300 pm in half log dilutions.

TABLE 2
IC₅₀ FOR SELECTED COMPOUNDS OF FORMULA A

| Compound No. | IC₅₀ (μM) |
|---|---|
| 5 | 5.94 |
| 6 | 0.54 |
| 7 | 9.6 |
| 35 | 3.46 |
| 36 | 2.34 |
| 37 | 2.61 |
| 38 | 0.3 |
| 39 | 1.4 |
| 40 | 3.53 |
| 41 | 1.79 |
| 42 | 0.08 |
| 44 | 1.53 |
| 45 | 8.75 |
| 46 | 1.23 |
| 67 | 2.5 |
| 68 | 0.872 |
| 72 | 2.07 |
| 75 | 1.11 |
| 77 | 1.23 |
| 79 | 4.67 |
| 80 | 2.73 |
| 81 | 3.2 |
| 85 | 0.73 |
| 86 | 0.1443 |
| 87 | 0.1580 |
| 88 | 5.9760 |
| 89 | 2.7222 |
| 94 | 2.4700 |
| 98 | 1.0190 |
| 101 | 0.0345 |
| 103 | 5.7080 |
| 107 | 6.5725 |
| 108 | 0.0685 |
| 109 | 0.0665 |
| 111 | 0.4325 |
| 112 | 0.1910 |

The compounds of this invention may be formulated neat or may be combined with one or more pharmaceutically acceptable carriers for administration. For example, solvents, diluents and the like, and may be administered orally in such forms as tablets, capsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like, or parenterally in the form of sterile injectable solution or suspension containing from about 0.05 to 5% suspending agent in an isotonic medium. Such pharmaceutical preparations may contain, for example, from about 0.05 up to about 90% of the active ingredient in combination with the carrier, more usually between about 5% and 60% by weight.

In some embodiments, the formulations are administered transdermally which includes all methods of administration across the surface of the body and the inner linings of body passages including epithelial and mucosal tissues. Such administration may be in the form of a lotion, cream, colloid, foam, patch, suspension, or solution.

The effective dosage of active ingredient employed may vary depending on the particular compound employed, the mode of administration and the severity of the condition being treated. However, in general, satisfactory results are obtained when the compounds of the invention are administered at a daily dosage of from about 0.5 to about 1000 mg/kg of animal body weight, optionally given in divided doses two to four times a day, or in sustained release form. For most large mammals the total daily dosage is from about 1 to 1000 mg, preferably from about 2 to 500 mg. Dosage forms suitable for internal use comprise from about 0.5 to 1000 mg of the active compound in intimate admixture with a solid or liquid pharmaceutically acceptable carrier. This dosage regimen may be adjusted to provide the optimal therapeutic response. For example, several divided doses may be administered daily or the dose may be proportionally reduced as indicated by the exigencies of the therapeutic situation.

The compounds of this invention may be administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, BHT and BHA.

The preferred pharmaceutical compositions from the standpoint of ease of preparation and administration are solid compositions, particularly tablets and hard-filled or liquid-filled capsules. Oral administration of the compounds is sometimes desirable.

In some cases it may be desirable to administer the compounds directly to the airways in the form of an aerosol.

The compounds of this invention may also be administered parenterally or intraperitoneally. Solutions or suspensions of these active compounds as a free base or pharmacologically acceptable salt may be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, polyol (e.g., glycerol, propylene glycol and liquid polyethylene glycol), suitable mixtures thereof, and vegetable oils.

For the treatment of cancer, the compounds of this invention may be administered in combination with other antitumor substances or with radiation therapy. These other substances or radiation treatments may be given at the same or at different times as the compounds of this invention. These combined therapies may effect synergy and result in improved efficacy. For example, the compounds of this invention may be used in combination with mitotic inhibitors such as taxol or vinblastine, alkylating agents such as cisplatin or cyclophosamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, and antiestrogens such as tamoxifen.

As used in accordance with this invention, the term “providing an effective amount of a compound” means either directly administering such compound, or administering a prodrug, derivative, or analog which will form an effective amount of the compound within the body.

Methods of administration of a pharmaceutical composition of the invention are not specifically restricted, and can be administered in various preparations depending on the age, sex, and symptoms of the patient. For example, tablets, pills, solutions, suspensions, emulsions, granules and capsules may be orally administered. Injection preparations may be administered individually or mixed with injection transfusions such as glucose solutions and amino acid solutions intravenously. If necessary, the injection preparations are administered singly intramuscularly, intracutaneously, subcutaneously or intraperitoneally. Suppositories may be administered into the rectum.

The amount of the compound of formula Ia contained in a pharmaceutical composition according to the present invention is not specifically restricted, however, the dose should be sufficient to treat, ameliorate, or reduce the targeted symptoms. The dosage of a pharmaceutical composition according to the present invention will depend on the method of use, the age, sex, and condition of the patient.

The present invention also provides methods of inhibition and treatment further comprising administering an additional inhibitor of a oncopprotein kinase of the Ras/Raf/MEK pathway.

The pharmaceutical compositions of the present invention may comprise the compound of the present invention alone or in combination with other oncoprotein kinase-inhibiting compounds or chemotherapeutic agents. Chemotherapeutic agents include, but are not limited to exemestane, formestane, anastrozole, letrozole, fadrozole, taxane and derivatives such as paclitaxel or docetaxel, encapsulated taxanes, CPT-11, camptothecin derivatives, anthracycline glycosides, e.g., doxorubicin, idarubicin, epirubicin, etoposide, navelbine, vinblastine, carboplatin, cisplatin, estramustine, celecoxib, tamoxifen, raloxifen, Sugen SU-5416, Sugen SU-6668, and Herceptin.

The following examples further describe and demonstrate features of the present invention. The examples are given solely for the purpose of illustration and are not to be construed as a limitation of the present invention.

### EXAMPLES

### Example 1

### N-(3-THIENO[3,2-D]PYRIMIDIN-4-YL-PHENYL)-3-TRIFLUOROMETHYL-BENZAMIDE (COMPOUND NO.1)

### Step 1: 3-Thieno[3,2-d]pyrimidin-4-yl-phenylamine

A stirred mixture of 4-chloro-thieno[3,2-d]pyrimidine (1.70 g, 10.0 mmol), 3-amino-benzeneboronic acid (1.60 g, 10.3 mmol), tetrakis(triphenylphosphine)palladium (0.35 g, 0.30 mmol) in 30 mL of 2.0 M sodium carbonate and 50 mL of DME was refluxed under a nitrogen atmosphere for 2 h, and the reaction was indicated complete by LCMS. The reaction mixture was cooled to room temperature, and diluted with 200 mL of ethyl acetate. The organic layer was washed with brine and dried over anhydrous sodium sulfate. The title product (2.8 g) was obtained by concentration and used in the next step without further purification.

### Step 2: N-(3-thieno[3,2-d]pyrimidin-4-yl-phenyl)-3-trifluoromethyl-benzamide

To the crude 3-thieno[3,2-d]pyrimidin-4-yl-phenylamine (2.8 g) from the previous step in methylene chloride was added 3-trifluoromethylbenzoyl chloride (2.8 g, 13 mmol) and triethylamine (3.6 mL, 26 mmol). The reaction mixture was stirred at room temperature for 16 h. The reaction mixture was concentrated and purified by flash chromatography (elution with 0-100% Ethyl acetate/Hexane) to give the title compound (3.3 g, 83% yield). MS: m/z 400 [M+H]

Compounds 2 and 3 were prepared following the procedure described for example 1 by using corresponding starting materials in Step 1.

| Compound | | |
|---|---|---|
| No. | Compound Name | MS, m/z |
| 2 | N-(3-thieno[2,3-d]pyrimidin-4-ylphenyl)-3- | 400 [M + H] |
| | (trifluoromethyl)benzamide | |
| 3 | N-[3-(7-methylthieno[3,2-d]pyrimidin-4- | 412 [M − H] |
| | yl)phenyl]-3-(trifluoromethyl)-benzamide | |

### Example 2

### ETHYL 4-(3-{[3-(TRIFLUOROMETHYL)BENZOYL]AMINO}PHENYL)THIENO[2,3-D]PYRIMIDINE-6-CARBOXYLATE (COMPOUND NO. 4)

### Step 1: 4-Chloro-thieno[2,3-d]pyrimidine-6-carboxylic acid

To a round bottom flask containing 2.0 M LDA in hexane (4.6 mL, 9.2 mmol) and 16 mL of THF cooled to −78° C. was added 4-chloro-thieno[2,3-d]pyrimidine (1.3 g, 7.6 mmol) dissolved in 9 mL of THF. The reaction mixture was stirred at −78° C. for 30 minutes, then CO₂ gas was bubbled through the reaction mixture for 1 hour. The reaction mixture was warmed to room temperature and quenched by adding ammonium chloride aqueous solution. The reaction mixture was partitioned between ethyl acetate and water. The aqueous layer was collected and neutralized to a pH around 2 by adding 2N hydrogen chloride. The title product (0.29 g, 18% yield) was obtained as a white solid by filtration. MS: m/z 169 [M—COOH]. ¹H NMR (CDCl₃): δ 9.1 (s,1H), 8.1 (s, 1H).

### Step 2: 4-(3-Amino-phenyl)-thieno[2,3-d]pyrimidine-6-carboxylic acid

Prepared according to the procedure of Step 1 of Example 1 using 4-chloro-thieno[2,3-d]pyrimidine-6-carboxylic acid. MS: m/z 272 [M+H]

### Step 3: 4-(3-Amino-phenyl)-thieno[2,3-d]pyrimidine-6-carboxylic acid ethyl ester

To the crude 4-(3-amino-phenyl)-thieno[2,3-d]pyrimidine-6-carboxylic acid of the previous step was added 3 drops of BF₃.Et₂O and 5 mL of ethanol, and the reaction mixture heated at 80° C. over night. The title product was obtained by concentration. MS: m/z 300 [M+H]

### Step 4: Ethyl 4-(3-{[3-(trifluoromethyl)benzoyl]amino}phenyl)thieno[2,3-d]pyrimidine-6-carboxylate

Prepared according to the procedure of Step 2, Example 1 from 4-(3-amino-phenyl)-thieno[2,3-d]pyrimidine-6-carboxylic acid ethyl ester. MS: m/z 472 [M+H]

### Example 3

### N-[3-(6-CYANO-2-PYRIDIN-4-YL-THIENO[3,2-B]PYRIDIN-7-YL)-PHENYL]-3-TRIFLUOROMETHYL-BENZAMIDE (COMPOUND NO. 5)

### Step 1: 7-chloro-2-pyridin-4-yl-thieno[3,2-b]pyridine-6-carbonitrile

A mixture of 7-chloro-2-iodo-thieno[3,2-b]pyridine-6-carbonitrile (prepared using the procedure described in J. Med. Chem. 2004, 47, 6666-6668) (200 mg, 0.62 mmol), pyridyl-4-boronic acid (77 mg, 0.62 mmol), tetrakis(triphenylphosphine)palladium (15 mg, 0.013 mmol) in 2 mL of 2.0 M sodium carbonate and 6 mL of DME was heated at 150° C. in a sealed tube in a microwave oven for 1000 seconds. The reaction mixture was concentrated and purified by ISCO (Combi-Flash) flash chromatography (elution with 0-100% Ethyl acetate/Hexane) to give the title compound (50 mg, 30% yield). MS: m/z 272 [M+H]

### Step 2: 7-(3-amino-phenyl)-2-pyridin-4-yl-thieno[3,2-b]pyridine-6-carbonitrile

A mixture of 7-chloro-2-pyridin-4-yl-thieno[3,2-b]pyridine-6-carbonitrile (50 mg, 0.18 mmol), 3-amino-benzeneboronic acid (28 mg, 0.18 mmol), tetrakis(triphenylphosphine)palladium (5 mg, 0.004 mmol) in 2 mL of 2.0 M sodium carbonate and 6 mL of DME was heated at 150° C. in a sealed tube in a microwave oven for 1000 seconds. The reaction mixture was concentrated and purified by ISCO (Combi-Flash) flash chromatography (elution with 0-100% Ethyl acetate/Hexane) to give the title compound (40 mg, 67% yield). MS: m/z 329 [M+H]

### Step 3: 7-(3-amino-phenyl)-2-pyridin-4-yl-thieno[3,2-b]pyridine-6-carbonitrile

N-[3-(6-cyano-2-pyridin-4-yl-thieno[3,2-b]pyridin-7-yl)-phenyl]-3-trifluoromethyl-benzamide (16.5 mg, 43% yield) was prepared according to the procedure of Step 2, Example 1 from 7-(3-amino-phenyl)-2-pyridin-4-yl-thieno[3,2-b]pyridine-6-carbonitrile. MS: m/z 501 [M+H]. ¹H NMR (DMSO-d6) δ 9.2 (s, 1H), 8.7 (d, 2H), 8.4 (s, 1H), 8.31(d,2H), 8.29 (s, 1H), 8.05 (m, 3H), 7.92 (d, 2H), 7.8 (t, 1H), 7.7 (t, 1H), 7.5 (d, 1H).

Compound Numbers 7, 9, 10, 12, 14, 18, 19, 21, 22, 24, 26, 27, and 31 were prepared following the procedure described for Example 3 by using corresponding starting materials in Step 3.

| Compound | | |
|---|---|---|
| No. | Compound Name | MS, m/z |
| 7 | N-[3-(6-cyano-2-pyridin-4-ylthieno[3,2-b]pyridin-7-yl)phenyl]-4- | 531 [M + H] |
| | methoxy-3-(trifluoromethyl)benzamide | |
| 9 | N-[3-(6-cyano-2-pyridin-4-ylthieno[3,2-b]pyridin-7-yl)phenyl]-4- | 515 [M + H] |
| | methyl-3-(trifluoromethyl)benzamide | |
| 10 | N-[3-(6-cyano-2-pyridin-4-ylthieno[3,2-b]pyridin-7-yl)phenyl]-4-fluoro- | 519 [M + H] |
| | 3-(trifluoromethyl)benzamide | |
| 12 | 4-chloro-N-[3-(6-cyano-2-pyridin-4-ylthieno[3,2-b]pyridin-7- | 535 [M + H] |
| | yl)phenyl]-3-(trifluoromethyl)benzamide | |
| 14 | 3,4-dichloro-N-[3-(6-cyano-2-pyridin-4-ylthieno[3,2-b]pyridin-7- | 499 [M − H] |
| | yl)phenyl]benzamide | |
| 18 | 3-chloro-N-[3-(6-cyano-2-pyridin-4-ylthieno[3,2-b]pyridin-7- | 465 [M − H] |
| | yl)phenyl]benzamide | |
| 19 | N-[3-(6-cyano-2-pyridin-3-ylthieno[3,2-b]pyridin-7-yl)phenyl]-4- | 529 [M − H] |
| | methoxy-3-(trifluoromethyl)benzamide | |
| 21 | N-[3-(6-cyano-2-pyridin-3-ylthieno[3,2-b]pyridin-7-yl)phenyl]-4- | 513 [M − H] |
| | methyl-3-(trifluoromethyl)benzamide | |
| 22 | N-[3-(6-cyano-2-pyridin-3-ylthieno[3,2-b]pyridin-7-yl)phenyl]-4-fluoro- | 517 [M − H] |
| | 3-(trifluoromethyl)benzamide | |
| 24 | 4-chloro-N-[3-(6-cyano-2-pyridin-3-ylthieno[3,2-b]pyridin-7- | 533 [M − H] |
| | yl)phenyl]-3-(trifluoromethyl)benzamide | |
| 26 | N-[3-(6-cyano-2-pyridin-3-ylthieno[3,2-b]pyridin-7-yl)phenyl]-3- | 499 [M − H] |
| | (trifluoromethyl)benzamide | |
| 27 | 3,4-dichloro-N-[3-(6-cyano-2-pyridin-3-ylthieno[3,2-b]pyridin-7- | 499 [M − H] |
| | yl)phenyl]benzamide | |
| 31 | 3-chloro-N-[3-(6-cyano-2-pyridin-3-ylthieno[3,2-b]pyridin-7- | 465 [M − H] |
| | yl)phenyl]benzamide | |

### EXAMPLE 4

### N-[3-(6-CYANO-2-PYRIDIN-4-YLTHIENO[3,2-B]PYRIDIN-7-YL)PHENYL]-N′-[3-(TRIFLUOROMETHYL)PHENYL]UREA (COMPOUND NO. 13)

A mixture of 7-(3-amino-phenyl)-2-pyridin-4-yl-thieno[3,2-b]pyridine-6-carbonitrile (Step 2, Example 3) (25 mg, 0.08 mmol) and 3-trifluoromethyl-phenylisocyanate (15 mg, 0.08 mmol) in 2 mL of THF was heated at 65° C. for 3 h. The reaction mixture was concentrated and purified by ISCO (Combi-Flash) flash chromatography (elution with 0-5% Methanol/Ethyl acetate) to give the title product (15 mg, 36% yield). MS: m/z: 516 [M+H]. ¹H NMR (DMSO-d6): δ 9.21 (d, 2H), 9.18 (s, 1H), 8.71 (d, 2H), 8.57 (s, 1H), 7.98 (d, 2H), 7.92 (d, 2H), 7.71 (d, 1H), 7.61 (m, 2H), 7.59 (m, 1H), 7.47 (d, 1H), 7.38 (d, 1H).

Compound Numbers 8, 11, 15, 16, 17, 20, 23, 25, 28, 29, and 30 were prepared following the procedure described for example 4 by using corresponding starting materials.

| Compound | | |
|---|---|---|
| No. | Compound Name | MS, m/z |
| 8 | N-[3-(6-cyano-2-pyridin-4-ylthieno[3,2-b]pyridin-7-yl)phenyl]-N′-[4- | 534 [M + H] |
| | fluoro-3-(trifluoromethyl)phenyl]urea | |
| 11 | N-[4-chloro-3-(trifluoromethyl)phenyl]-N′-[3-(6-cyano-2-pyridin-4- | 550 [M + H] |
| | ylthieno[3,2-b]pyridin-7-yl)phenyl]urea | |
| 15 | N-[3-(6-cyano-2-pyridin-4-ylthieno[3,2-b]pyridin-7-yl)phenyl]-N′-(3,4- | 474 [M − H] |
| | dimethylphenyl)urea | |
| 16 | N-(3-chlorophenyl)-N′-[3-(6-cyano-2-pyridin-4-ylthieno[3,2-b]pyridin- | 480 [M − H] |
| | 7-yl)phenyl]urea | |
| 17 | N-(4-chlorophenyl)-N′-[3-(6-cyano-2-pyridin-4-ylthieno[3,2-b]pyridin- | 480 [M − H] |
| | 7-yl)phenyl]urea | |
| 20 | N-[3-(6-cyano-2-pyridin-3-ylthieno[3,2-b]pyridin-7-yl)phenyl]-N′-[4- | 532 [M − H] |
| | fluoro-3-(trifluoromethyl)phenyl]urea | |
| 23 | N-[4-chloro-3-(trifluoromethyl)phenyl]-N′-[3-(6-cyano-2-pyridin-3- | 548 [M − H] |
| | ylthieno[3,2-b]pyridin-7-yl)phenyl]urea | |
| 25 | N-[3-(6-cyano-2-pyridin-3-ylthieno[3,2-b]pyridin-7-yl)phenyl]-N′-[3- | 514 [M − H] |
| | (trifluoromethyl)phenyl]urea | |
| 28 | N-[3-(6-cyano-2-pyridin-3-ylthieno[3,2-b]pyridin-7-yl)phenyl]-N′-(3,4- | 474 [M − H] |
| | dimethylphenyl)urea | |
| 29 | N-(3-chlorophenyl)-N′-[3-(6-cyano-2-pyridin-3-ylthieno[3,2-b]pyridin- | 480 [M − H] |
| | 7-yl)phenyl]urea | |
| 30 | N-(4-chlorophenyl)-N′-[3-(6-cyano-2-pyridin-3-ylthieno[3,2-b]pyridin- | 480 [M − H] |
| | 7-yl)phenyl]urea | |

### Example 5

### N-[3-(6-PYRIDIN-4-YL-THIENO[3,2-D]PYRIMIDIN-4-YL)-PHENYL]-3-TRIFLUOROMETHYLBENZAMIDE (COMPOUND NO. 6)

### Step 1: 3-(3-trifluoromethyl-benzoylamino)-benzeneboronic acid

A mixture of 3-amino-benzeneboronic acid (0.93 g, 6.0 mmol), 3-trifluoro-methyl-benzoyl chloride (1.3 g, 6.3 mmol) and triethylamine (1.7 mL, 12 mmol) in 25 mL of methylene chloride was stirred at room temperature overnight. The reaction mixture was concentrated and purified by ISCO (Combi-Flash) flash chromatography (elution with 0-10% Methanol/CH₂Cl₂) to give the title compound (1.5 g, 81% yield) as a white solid. MS: m/z 310 [M+H]

### Step 2: 4-chloro-6-iodo-thieno[3,2-d]pyrimidine

To a round bottom flask containing 2.0 M LDA in hexane (4.6 mL, 9.2 mmol) and 16 mL of THF cooled to −78° C., was added 4-chloro-thieno[3,2-d]pyrimidine (1.3 g, 7.6 mmol) dissolved in 9 mL of THF. After stirring at −78° C. for 20 minutes, 12 (2.3 g, 9.0 mmol) in 5 mL of THF was added. The reaction mixture was warmed to room temperature and stirred at room temperature for 30 minutes. The reaction was quenched by adding 200 mL of chloroform. The reaction mixture was partitioned between chloroform and water. The organic layer was collected and the aqueous layer was extracted with 100 mL of chloroform. The combined organic layers were washed with 100 mL aqueous sodium thiosulfate twice and 200 mL of brine. The organic layer was concentrated and purified by ISCO (Combi-Flash) flash chromatography (elution with 0-100% Ethyl acetate/Hexane) to give the title compound (1.6 g, 69% yield). ¹H NMR (CDCl₃): δ 9.0 (s, 1H), 7.8 (s, 1H).

### Step 3: 4-Chloro-6-pyridin-4-yl-thieno[3,2-d]pyrimidine

Prepared according to the procedure of Step 1, Example 3 from 4-chloro-6-iodo-thieno[3,2-d]pyrimidine. MS: m/z 248 [M+H]

### Step 4: N-[3-(6-pyridin-4-yl-thieno[3,2-d]pyrimidin-4-yl)-phenyl]-3-trifluoromethyl-benzamide

Prepared according to the procedure of Step 2, Example 3 from 4-chloro-6-pyridin-4-yl-thieno[3,2-d]pyrimidine and 3-(3-trifluoromethyl-benzoylamino)-benzeneboronic acid. MS: m/z 477 [M+H]

Compound Numbers 61-63, and 68-84 were prepared following the procedure described for example 5 by using corresponding starting materials in step 2.

| Compound | | |
|---|---|---|
| No. | Compound Name | MS, m/z |
| 61 | N-[3-(5-cyano-2-pyridin-4-ylthieno[2,3-b]pyridin-4-yl)phenyl]-3- | 501 [M + H] |
| | (trifluoromethyl)benzamide | |
| 62 | N-[3-(5-cyano-2-pyridin-3-ylthieno[2,3-b]pyridin-4-yl)phenyl]-3- | 499 [M − H] |
| | (trifluoromethyl)benzamide | |
| 63 | N-[3-(6-pyridin-3-ylthieno[3,2-d]pyrimidin-4-yl)phenyl]-3- | 477 [M + H] |
| | (trifluoromethyl)benzamide | |
| 68 | N-(3-{6-[4-(acetylamino)phenyl]thieno[2,3-d]pyrimidin-4-yl}phenyl)-3- | 533.1 [M + H] |
| | (trifluoromethyl)benzamide | |
| 69 | N-{3-[6-(4-aminophenyl)thieno[2,3-d]pyrimidin-4-yl]phenyl}-3- | 491.1 [M + H] |
| | (trifluoromethyl)benzamide | |
| 70 | N,N-dimethyl-4-[4-(3-{[3- | 547.1 [M + H] |
| | (trifluoromethyl)benzoyl]amino}phenyl)thieno[2,3-d]pyrimidin-6- | |
| | yl]benzamide | |
| 71 | N,N-dimethyl-3-[4-(3-{[3- | 545.1 [M − H] |
| | (trifluoromethyl)benzoyl]amino}phenyl)thieno[2,3-d]pyrimidin-6- | |
| | yl]benzamide | |
| 72 | N-(3-{6-[3-(acetylamino)phenyl]thieno[2,3-d]pyrimidin-4-yl}phenyl)-3- | 531.1 [M − H] |
| | (trifluoromethyl)benzamide | |
| 73 | N-(3-{6-[3-(dimethylamino)phenyl]thieno[2,3-d]pyrimidin-4- | 519.1 [M + H] |
| | yl}phenyl)-3-(trifluoromethyl)benzamide | |
| 74 | N-{3-[6-(3-aminophenyl)thieno[2,3-d]pyrimidin-4-yl]phenyl}-3- | 491.1 [M + H] |
| | (trifluoromethyl)benzamide | |
| 75 | N-[3-(6-pyridin-3-ylthieno[2,3-d]pyrimidin-4-yl)phenyl]-3- | 475.1 [M − H] |
| | (trifluoromethyl)benzamide | |
| 76 | N-{3-[6-(6-methoxypyridin-3-yl)thieno[2,3-d]pyrimidin-4-yl]phenyl}-3- | 507.1 [M + H] |
| | (trifluoromethyl)benzamide | |
| 77 | N-{3-[6-(4-cyanophenyl)thieno[2,3-d]pyrimidin-4-yl]phenyl}-3- | 499.1 [M − H] |
| | (trifluoromethyl)benzamide | |
| 78 | N-[3-(6-phenylthieno[2,3-d]pyrimidin-4-yl)phenyl]-3- | 476.1 [M + H] |
| | (trifluoromethyl)benzamide | |
| 79 | N-{3-[6-(4-chlorophenyl)thieno[2,3-d]pyrimidin-4-yl]phenyl}-3- | 508.3 [M − H] |
| | (trifluoromethyl)benzamide | |
| 80 | N-{3-[6-(4-acetylphenyl)thieno[2,3-d]pyrimidin-4-yl]phenyl}-3- | 516.1 [M − H] |
| | (trifluoromethyl)benzamide | |
| 81 | N-{3-[6-(3-acetylphenyl)thieno[2,3-d]pyrimidin-4-yl]phenyl}-3- | 516.1 [M − H] |
| | (trifluoromethyl)benzamide | |
| 82 | N-{3-[6-(4-methoxyphenyl)thieno[2,3-d]pyrimidin-4-yl]phenyl}-3- | 506.1 [M + H] |
| | (trifluoromethyl)benzamide | |
| 83 | N-{3-[6-(4-methylphenyl)thieno[2,3-d]pyrimidin-4-yl]phenyl}-3- | 490.1 [M + H] |
| | (trifluoromethyl)benzamide | |
| 84 | N-{3-[6-(3-methylphenyl)thieno[2,3-d]pyrimidin-4-yl]phenyl}-3- | 490 [M + H] |
| | (trifluoromethyl)benzamide | |

### Example 6

### N-[3-(6-PYRIDIN-4-YL-THIENO[2,3-D]PYRIMIDIN-4-YL)-PHENYL]-3-TRIFLUOROMETHYL-BENZAMIDE (COMPOUND NO. 42)

### Step 1: 6-pyridin-4-yl-1H-thieno[2,3-d]pyrimidin-4-one

A reaction mixture of 2-amino-5-pyridin-4-yl-thiophene-3-carboxylic acid ethyl ester (prepared according to the procedure described in PCT Int. Appl. WO 2004/041813 A1) (6.4 g, 26 mmol), formamidine acetate (8.1 g, 77 mmol) in 100 mL of methoxyethanol was refluxed for 6 hours. The reaction mixture was cooled to room temperature and then 50 mL of water was added. After filtration and washing with 200 mL of water and 50 mL of ether, the title compound (5.2 g, 88% yield) was obtained as a white solid. MS: m/z 230 [M+H]. ¹H NMR (DMSO-d₆): δ 9.0 (s, 1H), 8.6 (d, 2H), 8.2 (s, 1H), 8.15 (s, 1H), 7.8 (d, 2H).

### Step 2: 4-chloro-6-pyridin-4-yl-thieno[2,3-d]pyrimidine

To 6-pyridin-4-yl-1H-thieno[2,3-d]pyrimidin-4-one (2.3 g, 1.0 mmol) was added 20 mL of thionyl chloride and 10 mL of DMF. The reaction mixture was refluxed for 4 hours and cooled to room temperature. The thionyl chloride and DMF were removed by rotary evaporation, and the residue triturated with ethyl acetate. After filtration and washing with ethyl acetate, methylene [chloride?] and ether, the title compound (2.3 g, 93% yield) was obtained as a solid. MS: m/z 248 [M+H]. ¹H NMR (DMSO-d₆): δ 9 (s, 1H), 8.7 (d, 2H), 8.5 (s, 1H), 8.2 (d, 2H).

### Step 3: 3-(6-Pyridin-4-yl-thieno[2,3-d]pyrimidin-4-yl)-phenylamine

The title compound (0.44 g, 67% yield) was prepared according to the procedure of Step 2, Example 3 from 4-Chloro-6-pyridin-4-yl-thieno[2,3-d]pyrimidine (0.53 g, 2.1 mmol). m/z 305 [M+H]

### Step 4: N-[3-(6-pyridin-4-yl-thieno[2,3-d]pyrimidin-4-yl )-phenyl]-3-trifluoromethyl-benzamide

The title compound (38 mg, 79% yield)) was prepared according to the procedure of Step 2, Example 1 from 3-(6-Pyridin-4-yl-thieno[2,3-d]pyrimidin-4-yl)-phenylamine 931 mg, 0.1 mmol). MS: m/z 477 [M+H]. ¹H NMR (DMSO-d₆): δ 10.8 (s, 1H), 9.22 (s, 1H), 8.71 (d, 2H), 8.53 (s, 1H), 8.48 (s, 1H), 8.37 (s, 1H), 8.34 (d, 1H), 8.0 (d, 1H), 7.95 (d, 1H), 7.88 (d, 2H), 7.88 (t, 1H), 7.83 (t, 1H), 7.66 (t, 1H)

Compound Numbers 35, 37, 38, 40, 43, 47, 49, 50, 51, 53, 55, 56, 60, and 85 were prepared following the procedure described for example 6 by using corresponding starting materials.

| Compound | | |
|---|---|---|
| No. | Compound Name | MS, m/z |
| 35 | 4-methoxy-N-[3-(6-pyridin-4-ylthieno[2,3-d]pyrimidin-4-yl)phenyl]-3- | 507 [M + H] |
| | (trifluoromethyl)benzamide | |
| 37 | 4-methyl-N-[3-(6-pyridin-4-ylthieno[2,3-d]pyrimidin-4-yl)phenyl]-3- | 491 [M + H] |
| | (trifluoromethyl)benzamide | |
| 38 | 4-fluoro-N-[3-(6-pyridin-4-ylthieno[2,3-d]pyrimidin-4-yl)phenyl]-3- | 495 [M + H] |
| | (trifluoromethyl)benzamide | |
| 39 | N-[4-chloro-3-(trifluoromethyl)phenyl]-N′-[3-(6-pyridin-4-ylthieno[2,3- | 526 [M + H] |
| | d]pyrimidin-4-yl)phenyl]urea | |
| 40 | 4-chloro-N-[3-(6-pyridin-4-ylthieno[2,3-d]pyrimidin-4-yl)phenyl]-3- | 511 [M + H] |
| | (trifluoromethyl)benzamide | |
| 43 | 3,4-dichloro-N-[3-(6-pyridin-4-ylthieno[2,3-d]pyrimidin-4- | 477 [M + H] |
| | yl)phenyl]benzamide | |
| 47 | 3-chloro-N-[3-(6-pyridin-4-ylthieno[2,3-d]pyrimidin-4- | 443 [M + H] |
| | yl)phenyl]benzamide | |
| 49 | N-[4-fluoro-3-(trifluoromethyl)phenyl]-N′-[3-(5-pyridin-4-ylthieno[2,3- | 510 [M + H] |
| | d]pyrimidin-4-yl)phenyl]urea | |
| 50 | 4-methyl-N-[3-(5-pyridin-4-ylthieno[2,3-d]pyrimidin-4-yl)phenyl]-3- | 491 [M + H] |
| | (trifluoromethyl)benzamide | |
| 51 | 4-fluoro-N-[3-(5-pyridin-4-ylthieno[2,3-d]pyrimidin-4-yl)phenyl]-3- | 495 [M + H] |
| | (trifluoromethyl)benzamide | |
| 53 | 4-chloro-N-[3-(5-pyridin-4-ylthieno[2,3-d]pyrimidin-4-yl)phenyl]-3- | 511 [M + H] |
| | (trifluoromethyl)benzamide | |
| 55 | N-[3-(5-pyridin-4-ylthieno[2,3-d]pyrimidin-4-yl)phenyl]-3- | 477 [M + H] |
| | (trifluoromethyl)benzamide | |
| 56 | 3,4-dichloro-N-[3-(5-pyridin-4-ylthieno[2,3-d]pyrimidin-4- | 477 [M + H] |
| | yl)phenyl]benzamide | |
| 60 | 3-chloro-N-[3-(5-pyridin-3-ylthieno[2,3-d]pyrimidin-4- | 443 [M + H] |
| | yl)phenyl]benzamide | |
| 85 | N-methyl-4-[4-(3-{[3-(trifluoromethyl)benzoyl]amino}- | 534 [M + H] |
| | phenyl)thieno[2,3-d]pyrimidin-6-yl]pyridine-2-carboxamide | |

### Example 7

### N-[4-FLUORO-3-(TRIFLUOROMETHYL)PHENYL]-N′-[3-(6-PYRIDIN-4-YLTHIENO[2,3- D]PYRIMIDIN-4-YL)PHENYL]UREA (COMPOUND NO. 36)

N-[4-fluoro-3-(trifluoromethyl)phenyl]-N′-[3-(6-pyridin-4-ylthieno[2,3-d]pyrimidin-4-yl)phenyl]urea (39 mg, 76% yield) was prepared according to the procedure of Example 4 from 3-(6-pyridin-4-yl-thieno[2,3-d]pyrimidin-4-yl)-phenylamine [Step 3, Example 6] (25 mg, 0.08 mmol) and 3-trifluoromethyl4-fluoro-phenylisocyanate. MS: m/z 510 [M+H]

Compound Numbers 39, 41, 44, 45, 46, 49, 52, 54, 56, 57, and 58 were prepared following the procedure described for example 7 by using corresponding starting materials.

| Compound | | |
|---|---|---|
| No. | Compound Name | MS, m/z |
| 39 | N-[4-chloro-3-(trifluoromethyl)phenyl]-N′-[3-(6-pyridin-4-ylthieno[2,3- | 526 [M + H] |
| | d]pyrimidin-4-yl)phenyl]urea | |
| 41 | N-[3-(6-pyridin-4-ylthieno[2,3-d]pyrimidin-4-yl)phenyl]-N′-[3- | 492 [M + H] |
| | (trifluoromethyl)phenyl]urea | |
| 44 | N-(3,4-dimethylphenyl)-N′-[3-(6-pyridin-4-ylthieno[2,3-d]pyrimidin-4- | 452 [M + H] |
| | yl)phenyl]urea | |
| 45 | N-(3-chlorophenyl)-N′-[3-(6-pyridin-4-ylthieno[2,3-d]pyrimidin-4- | 458 [M + H] |
| | yl)phenyl]urea | |
| 46 | N-(4-chlorophenyl)-N′-[3-(6-pyridin-4-ylthieno[2,3-d]pyrimidin-4- | 458 [M + H] |
| | yl)phenyl]urea | |
| 49 | N-[4-fluoro-3-(trifluoromethyl)phenyl]-N′-[3-(5-pyridin-4-ylthieno[2,3- | 510 [M + H] |
| | d]pyrimidin-4-yl)phenyl]urea | |
| 52 | N-[4-chloro-3-(trifluoromethyl)phenyl]-N′-[3-(5-pyridin-4-ylthieno[2,3- | 526 [M + H] |
| | d]pyrimidin-4-yl)phenyl]urea | |
| 54 | N-[3-(5-pyridin-4-ylthieno[2,3-d]pyrimidin-4-yl)phenyl]-N′-[3- | 492 [M + H] |
| | (trifluoromethyl)phenyl]urea | |
| 56 | 3,4-dichloro-N-[3-(5-pyridin-4-ylthieno[2,3-d]pyrimidin-4- | 477 [M + H] |
| | yl)phenyl]benzamide | |
| 57 | N-(3,4-dimethylphenyl)-N′-[3-(5-pyridin-4-ylthieno[2,3-d]pyrimidin-4- | 452 [M + H] |
| | yl)phenyl]urea | |
| 58 | N-(3-chlorophenyl)-N′-[3-(5-pyridin-4-ylthieno[2,3-d]pyrimidin-4- | 458 [M + H] |
| | yl)phenyl]urea | |

### Example 8

### TERT-BUTYL 4-HYDROXY-4-[4-(3-{[3-(TRIFLUOROMETHYL)BENZOYL]AMINO}PHENYL)THIENO[2,3-D]PYRIMIDIN-6-YL]PIPERIDINE-1-CARBOXYLATE (COMPOUND NO. 64)

### Step 1: 4-(4-chloro-thieno[2,3-d]pyrimidin-6-yl)-4-hydroxy-piperidine-1-carboxylic acid tert-butyl ester

To 4-chloro-thieno[2,3-d]pyrimidine (0.85 g, 5.0 mmol) in 25 mL of THF at −78° C. was added 2.0 M LDA hexane solution (3.0 mL, 6.0 mmol). The reaction mixture was stirred at −78° C. for 1 hour. Then 4-oxo-piperidine-1-carboxylic acid tert-butyl ester (0.80 g, 4.0 mmol) in 3 mL of THF was added. The reaction mixture was stirred at −78° C. for 3 hours and quenched by adding aqueous ammonium chloride.

The reaction mixture was extracted with ethyl acetate (3×100 mL) and the combined organic layers were washed with brine. The organic layer was collected and concentrated and purified by ISCO (Combi-Flash) flash chromatography (elution with 0-40% Ethyl acetate/Hexane) to give the title compound (1.3 g, 85% yield). MS: m/z 370 [M+H]

### Step 2: tert-Butyl 4-hydroxy-4-[4-(3-{[3-(trifluoromethyl)benzoyl]amino}phenyl)thieno[2,3-d]pyrimidin-6-yl]piperidine-1-carboxylate

The title compound (427 mg, 79% yield) was prepared according to the procedure of Step 2, Example 3 from 4-(4-chloro-thieno[2,3-d]pyrimidin-6-yl)-4-hydroxy-piperidine-1-carboxylic acid tert-butyl ester and 3-(3-trifluoromethyl-benzoylamino)-benzeneboronic acid (Step 1, Example 5) MS: m/z 599 [M+H]

### Example 9

### N-{3-[6-(4-HYDROXYPIPERIDIN-4-YL)THIENO[2,3-D]PYRIMIDIN-4-YL]PHENYL}-3-(TRIFLUOROMETHYL)BENZAMIDE (COMPOUND NO. 65)

To tert-butyl 4-hydroxy-4-[4-(3-{[3-(trifluoromethyl)benzoyl]amino}phenyl)thieno[2,3-d]pyrimidin-6-yl]piperidine-1-carboxylate (196 mg, 0.328 mmol) in 5 mL of methylene chloride at 0° C. was added 1 mL of TFA. The reaction mixture was stirred at 0° C. for 2 hours and then concentrated. The title compound (126 mg, 77.2% yield) was obtained by reverse phase chromatography purification. MS: m/z 497 [M−H]

N-{3-[6-(1,2,3,6-tetrahydropyridin-4-yl)thieno[2,3-d]pyrimidin-4-yl]phenyl}-3-(trifluoromethyl)benzamide 67 was prepared following the procedure described for example 9 by using corresponding starting materials. MS: m/z 481 [M+H]

### Example 10

### TERT-BUTYL 4-[4-(3-{[3-(TRIFLUOROMETHYL)BENZOYL]AMINO}-PHENYL)THIENO[2,3-D]PYRIMIDIN-6-YL]-3,6-DIHYDROPYRIDINE-1(2H)-CARBOXYLATE (COMPOUND NO. 66)

To tert-butyl-4-hydroxy-4-[4-(3-{[3-(trifluoromethyl)benzoyl]amino}phenyl)thieno[2,3-d]pyrimidin-6-yl]piperidine-1-carboxylate (194 mg, 0.324 mmol) in 5 mL of methylene chloride was added methanesulfonyl chloride (0.13 mL, 1.7 mmol) and pyridine (0.14 mL, 1.7 mmol). The reaction mixture was stirred at room temperature for 3 hours and then concentrated. The title compound (22 mg, 12% yield) was purified by reverse phase chromatography. MS: m/z 581 [M+H].

### Example 11

### N-[3-(6-{2-[(2-MORPHOLIN-4-YLETHYL)AMINO]PYRIDIN-4-YL}THIENO[2,3-D]PYRIMIDIN-4-YL)PHENYL]-3-(TRIFLUOROMETHYL)BENZAMIDE (COMPOUND NO. 86)

### Step 1: 4-(3-Nitro-phenyl)-6-pyridin-4-yl-thieno[2,3-d]pyrimidine

To 4-chloro-6-pyridin-4-yl-thieno[2,3-d]pyrimidine (4.9 g, 19.8 mmol), were added Pd(PPh₃)₄ (1.15 g, 0.99 mmol), 2M K₂CO₃ de-gassed solution (30 ml, 60 mmol) and de-gassed DME (100 ml). The mixture was stirred at room temperature for 30 minutes; then 3-nitrophenylboronic acid (5 g, 29.8 mmol) was added. The reaction was heated at 85° C. overnight and then DME was evaporated. The residue was diluted with water and extracted with Ethyl Acetate+10% Methanol. was obtained as a light brown solid in 60% yield (4 g). ¹H (300 MHz, DMSO-d₆): δ 9.27(s, 1H); 8.80(dd, 1H); 8.72(m, 2H); 8.53(ddd, 1H); 8.48(ddd, 1H); 8.47(s, 1H); 7.98-7.91(m, 3H).

### Step 2: 4-(3-Nitro-phenyl)-6-(1-oxy-pyridin-4-yl)-thieno[2,3-d]pyrimidine

To 4-(3-nitro-phenyl)-6-pyridin-4-yl-thieno[2,3-d]pyrimidine (100 mg, 0.3 mmol), were added mCPBA (88 mg, 0.36 mmol) and CHCl₃ (10 ml). The mixture was refluxed for 2.5 h, then it was diluted with saturated NaHCO₃ solution and extracted with CH₂Cl₂+10% Methanol [10% methanol in methylene chloride?]. The crude product was used in next step without further purification (quantitative yield).

### Step 3: 6-(2-Chloro-pyridin-4-yl)-4-(3-nitro-phenyl)-thieno[2,3-d]pyrimidine

To 4-(3-nitro-phenyl)-6-(1-oxy-pyridin-4-yl)-thieno[2,3-d]pyrimidine, POCl₃ (5 ml) was added and the mixture was refluxed for 2 h. POCl₃ was evaporated and the residue was poured in saturated NaHCO₃ solution and extracted with CH₂Cl₂+10% Methanol [10% methanol in methylene chloride?]. The obtained crude product was purified by a silica gel column chromatography (eluent gradient from CH₂Cl₂ to 20:1 CH₂Cl₂-Methanol). The title compound was obtained as a white solid in 38% yield (42 mg). ¹H (300 MHz, DMSO-d₆): δ 9.28(s, 1H); 8.79(dd, 1H); 8.57(s, 1H); 8.56-8.47(m, 3H); 8.17(m, 1H); 7.95(dd, 1H); 7.92(dd, 1H).

### Step 4: 3-[6-(2-chloro-pyridin-4-yl)-thieno[2, 3-d]pyrimidin-4-yl]-phenylamine

To a stirred solution of 6-(2-chloro-pyridin-4-yl)-4-(3-nitro-phenyl)-thieno[2,3-d]pyrimidine (1 g, 2.72 mmol) in 1:1 Methanol-H₂O (300 ml) at 100° C., Na₂S₂O₄ was added (4.73 g, 27.2 mmol, 10 eq.) in portions over ten minutes. After stirring at 100° C. overnight, the solvent was evaporated. The crude was purified by silica gel column chromatography (eluent gradient from CH₂Cl₂ to 10:1 CH₂Cl₂-Methanol) affording title compound in 23% yield (210 mg). ¹H (300 MHz, DMSO-d₆): δ 9.15(s, 1H); 8.52(d, 1H); 8.42(s, 1H); 8.13(d, 1H); 7.90(dd, 1H); 7.32-7.19(m, 3H); 6.82(d, 1H); 5.63(s br, 2H).

### Step 5: N-{3-[6-(2-chloro-pyridin-4-yl)-thieno[2,3-d]pyrimidin-4-yl]-phenyl}-3-trifluoromethyl-benzamide

To a solution of 3-[6-(2-Chloro-pyridin-4-yl)-thieno[2,3-d]pyrimidin-4-yl]-phenylamine (210 mg, 0.621 mmol) and pyridine (100 μl, 1.24 mmol, 2 eq.) in CH₂Cl₂ (2 ml) was added 3-trifluoromethyl-benzoyl chloride (94 μl, 0.621 mmol, 1 eq.) at 0° C. dropwise. The resulting mixture was stirred at room temperature overnight, diluted with water and extracted with CH₂Cl₂. The crude product was used in the next step without further purification (quantitative yield).

### Step 6: N-[3-(6-{2-[(2-morpholin-4-ylethyl)amino]pyridin-4-yl}thieno[2,3-d]pyrimidin-4-yl)phenyl]-3-(trifluoromethyl)benzamide

Crude N-{3-[6-(2-chloro-pyridin-4-yl)-thieno[2,3-d]pyrimidin-4-yl]-phenyl}-3-trifluoro-methyl-benzamide was suspended in anhydrous NMP (4 mL) and (2-morpholin-4-ylethyl)amine (3 mL) was added. The reaction mixture was heated at 170° C. in a microwave oven for 40 minutes, then an additional amount of amine (2mL) was added and the reaction mixture was heated under microwave irradiation. 4/5 cycles were required to force the reaction to completion (including the addition of amine). NMP was removed by distillation under vacuum and the crude was firstly purified on silica gel (Ethyl acetate and 1:1 Ethyl acetate:Methanol) and then by preparative HPLC (5%-100% acetonitrile) led to 81.9 mg (22% yield) of title compound as a yellow solid. ¹H (300 MHz, DMSO-d₆+TFA): δ 10.76(s, 1H); 9.25(s, 1H); 8.56(s, 1H); 8.49(s, 1H); 8.36(s, 1H); 8.33(d, 1H); 8.18(d, 1H); 8.01(m, 2H); 7.87(d, 1H); 7.82(dd, 1H); 7.67(dd, 1H); 7.49(dd, 1H); 7.26(m, 2H); 3.85(m, 6H); 3.46-3.29 (m, 6H).

N-{3-[6-(2-{[3-(1H-imidazol-1-yl)propyl]amino}pyridin-4-yl)thieno[2,3-d]pyrimidin-4-yl]phenyl}-3-(trifluoromethyl)benzamide 87 was prepared following the procedure described for example 11 by using corresponding starting materials. MS: m/z 600 [M+H].

### Example 12

### 4-(3-{[3-(TRIFLUOROMETHYL)BENZOYL]AMINO}PHENYL)THIENO[2,3-D]PYRIMIDINE-6-CARBOXAMIDE (COMPOUND NO. 89)

### Step 1: 4-chlorothieno[2,3-d]pyrimidine-6-carboxylic acid

To a cold (−78° C.) 2M solution of lithium diisopropylamide in THF/heptane (9.2 mL, 18.4 mmol) in THF (32 mL) was added dropwise solution of 4-chloro[2,3-d]pyrimidine (2.6 g, 15.2 mmol) in THF (25 mL) over a period of 30 minutes and stirred at −78° C. for 20 minutes, and then CO₂ gas was bubbled for 1 hour maintaining temperature at −78° C. The mixture was allowed to warm to room temperature and stirred overnight. The clear red solution was quenched with saturated solution of ammonium chloride, diluted with ethyl acetate and two layers were separated. The aqueous layer was acidified with 2N hydrochloric acid to pH 2. The solid was collected by filtration, washed with water and dried to yield 2.5 g (78%) of title compound as a beige solid (mp>265° C.). MS: m/z 213.0 [M−H].

### Step 2: 4-chlorothieno[2,3-d]pyrimidine-6-carbonyl chloride

A mixture of 4-chlorothieno[2,3-d]pyrimidine-6-carboxylic acid (1.22 g, 5.7 mmol) and oxalyl chloride (3 mL, 34.4 mmol) in methylene chloride (35 mL) was heated under reflux for 3.5 hours. The reaction mixture was cooled and evaporated to dryness, and the residue was evaporated twice from toluene. The residue was dissolved in methylene chloride, filtered, and filtrate was evaporated to dryness to yield 1.23 g (95%) of title compound as a white solid which was used as is in the next step.

### Step 3: 4-chlorothieno[2,3-d]pyrimidine-6-carboxamide

To a cold (0-5° C.) solution of 4-chlorothieno[2,3-d]pyrimidine-6-carbonyl chloride (0.4 g, 1.71 mmol) in methylene chloride (6 mL) was bubbled NH₃ gas for about 2 minutes, and the resulting mixture was stirred at 5° C. for 20 minutes. The solvent was evaporated to dryness and the residue was treated with water, the solid that formed collected by filtration, washed with water and dried to yield 0.36 g (98%) of title compound as a white solid, which was used without further purification in the next step. MS: m/z 214.0 [M+H].

### Step 4: 4-(3-{[3-(trifluoromethyl)benzoyl]amino}phenyl)thieno[2,3-d]pyrimidine-6-carboxamide

A mixture of 4-chlorothieno[2,3-d]pyrimidine-6-carboxamide (0.12 g, 0.56 mmol), (3-{[3-(trifluoromethyl)benzoyl]amino}phenyl)boronic acid (0.38 g, 1.2 mmol), tetrakis(triphenylphosphine) palladium (0) (45 mg) and saturated solution of sodium bicarbonate (3 mL) in DME (9 mL) was heated in a microwave oven at 100° C. for 10 minutes. The mixture was filtered through a pad of Celite, washed with DME and the filtrate was evaporated to remove DME. The solid was collected by filtration, washed with water and dried. The solid was suspended in 10% methanol in methylene chloride (25 mL), stirred and collected by filtration, washed with 10% methanol in methylene chloride and dried to yield 0.13 g of the title compound as a white solid (mp 275-278° C.). MS: m/z 441.1 [M−H].

### Example 13

### N-[3-(6-CYANOTHIENO[2,3-D]PYRIMIDIN-4-YL)PHENYL]-3-(TRIFLUORO-METHYL)BENZAMIDE (COMPOUND NO. 88)

### Step1: 4-chlorothieno[2,3-d]pyrimidine-6-carbonitrile

A mixture of 4-chlorothieno[2,3-d]pyrimidine-6-carboxamide (0.32 g, 1.37 mmol) and cyanuric chloride in DMF (4 mL) was stirred and heated at 55° C. for 5 minutes. The mixture was cooled, ice was added and solid was collected by filtration, washed with water and dried. The crude product was purified by silica gel flash chromatography (Methanol/CH₂Cl₂) to yield 0.17 g (63%) of title compound as a white solid (mp=185-190° C.). MS: m/z 195.0 [M−H].

### Step 2: N-[3-(6-cyanothieno[2,3-d]pyrimidin-4-yl)phenyl]-3-(trifluoromethyl)benzamide

The title compound was prepared according to the procedure of Step 4, Example 12 from 4-chlorothieno[2,3-d]pyrimidine-6-carbonitrile. MP: 210-212° C., MS: m/z 423.2 [M−H].

### Example 14

### N-(3-(7H-PYRROLO[2,3-D]PYRIMIDIN-4-YL)PHENYL)-3-(TRIFLUOROMETHYL)BENZAMIDE (COMPOUND NO. 90)

A mixture of 3-(3-(trifluoromethyl)benzamido)phenylboronic acid (6 g, 19.5 mmol), 4-chloro-7H-pyrrolo[2,3-d]pyrimidine (3 g, 19.5 mmol), Tetrakispalladium triphenylphosphine (0.2.2 g, 1.95 mmol), and sodium carbonate (4.1 g, 39 mmol) in DMF (60 mL)/water (20 mL) were stirred 80° C. for 3 days. The reaction mixture was concentrated and extracted with water and Ethyl acetate. The organics were concentrated and the crude product was purified by flash chromatography (CH₂Cl₂:Ethyl acetate) to give the title compound (3.6 g, 48% yield). MS: m/z 383.2 [M+H].

### Example 15

### N-(3-(7-(3-(DIMETHYLAMINO)PROPYL)-7H-PYRROLO[2,3-D]PYRIMIDIN-4-YL)PHENYL)-3-(TRIFLUOROMETHYL)BENZAMIDE (COMPOUND NO. 91)

A mixture of N-(3-(7H-pyrrolo[2,3-d]pyrimidin-4-yl)phenyl)-3-(trifluoromethyl)-benzamide (0.15 g, 0.4 mmol) and NaH 60% in mineral oil (35 mg, 0.88 mmol) in DMF (3 mL) were stirred for 30 min. To this was added 3-chloro-N,N-dimethylpropan-1-amine hydrochloride (68 mg, 0.43 mmol) and the mixture was stirred at 80° C. for 12 hours. The reaction mixture was purified by reverse phase chromatography to give title compound (118 mg, 63% yield). MS: m/z 468.3 [M+H].

Compound Numbers 93, 94, 95, 96, 97, 98, 102 and 106 were prepared following the procedure described for example 15 using the appropriate alkylating agent.

| Compound | | |
|---|---|---|
| No. | Compound Name | MS, m/z |
| 93 | N-[3-(7-benzyl-7H-pyrrolo[2,3-d]pyrimidin-4-yl)phenyl]-3- | 473.2 [M + H] |
| | (trifluoromethyl)benzamide | |
| 94 | N-{3-[7-(2-cyanobenzyl)-7H-pyrrolo[2,3-d]pyrimidin-4-yl]phenyl}-3- | 498.1 [M + H] |
| | (trifluoromethyl)benzamide | |
| 95 | N-{3-[7-(3-cyanobenzyl)-7H-pyrrolo[2,3-d]pyrimidin-4-yl]phenyl}-3- | 498.1 [M + H] |
| | (trifluoromethyl)benzamide | |
| 96 | N-{3-[7-(4-cyanobenzyl)-7H-pyrrolo[2,3-d]pyrimidin-4-yl]phenyl}-3- | 498.1 [M + H] |
| | (trifluoromethyl)benzamide | |
| 97 | N-{3-[7-(pyridin-2-ylmethyl)-7H-pyrrolo[2,3-d]pyrimidin-4-yl]phenyl}- | 474.1 [M + H] |
| | 3-(trifluoromethyl)benzamide | |
| 98 | N-{3-[7-(pyridin-4-ylmethyl)-7H-pyrrolo[2,3-d]pyrimidin-4-yl]phenyl}- | 474.1 [M + H] |
| | 3-(trifluoromethyl)benzamide | |
| 102 | N-{3-[7-(1-methylpiperidin-4-yl)-7H-pyrrolo[2,3-d]pyrimidin-4- | 480.4 [M + H] |
| | yl]phenyl}-3-(trifluoromethyl)benzamide | |
| 106 | N-{3-[7-(3-formylbenzyl)-7H-pyrrolo[2,3-d]pyrimidin-4-yl]phenyl}-3- | 501.3 [M + H] |
| | (trifluoromethyl)benzamide | |

### Example 16

### N-{3-[7-(METHYLSULFONYL)-7H-PYRROLO[2,3-D]PYRIMIDIN-4-YL]PHENYL}-3-(TRIFLUOROMETHYL)BENZAMIDE (COMPOUND NO. 104)

A mixture of N-(3-(7H-pyrrolo[2,3-d]pyrimidin-4-yl)phenyl)-3-(trifluoromethyl)-benzamide (0.1 g, 0.26 mmol) and NaH 60% in mineral oil (26 mg, 0.65 mmol) in DMF (2 mL) were stirred for 30 min. To this was added methanesulfonyl chloride (0.025 mL, 0.31 mmol) and the mixture was stirred at 23° C. for 3 hours. Solvent was removed in vacuo, and the resulting residue was purified by reverse phase chromatography to give the title compound. (11 mg, 9.1% yield). MS: m/z 461.2 [M+H].

N-(3-{7-[(4-methylphenyl)sulfonyl]-7H-pyrrolo[2,3-d]pyrimidin-4-yl}phenyl)-3-(trifluoromethyl)benzamide 92 was prepared following the procedure described for example 16, by using the corresponding starting materials. MS: m/z 537.1 [M+H].

### Example 17

### N-(3-(7-(4-(MORPHOLINOMETHYL)BENZYL)-7H-PYRROLO[2,3-D]PYRIMIDIN-4-YL)PHENYL)-3-(TRIFLUOROMETHYL)BENZAMIDE (COMPOUND NO. 99)

### Step 1: 4-(morpholinomethyl)benzyl 4-methylbenzenesulfonate

(4-(morpholinomethyl)phenyl)methanol (0.108 g, 0.52 mmol) and NaH 60% in mineral oil (13 mg, 0.52 mmol) in THF (2 mL) were stirred for 30 min. Tosyl Chloride (60 mg, 0.52 mmol) was added to this solution and stirred at room temperature for 2 hours. The crude reaction mixture was used in the next step.

### Step 2: N-(3-(7-(4-(morpholinomethyl)benzyl)-7H-pyrrolo[2,3-d]pyrimidin-4-yl)phenyl)-3-(trifluoromethyl)benzamide

The title compound was prepared according to the procedure of example 91 from 4-(morpholinomethyl)benzyl-4-methylbenzenesulfonate and N-(3-(7H-pyrrolo[2,3-d]pyrimidin-4-yl)phenyl)-3-(trifluoromethyl)benzamide. MS: m/z 572.2 [M+H].

N-(3-{7-[4-(2-methyl-1H-imidazol-1-yl)benzyl]-7H-pyrrolo[2,3-d]pyrimidin-4-yl}phenyl)-3-(trifluoromethyl)benzamide 100 was prepared following the procedure described for example 17 using the corresponding starting materials. MS: m/z 553.2 [M+H].

### Example 18

### N-[3-(6-PYRIDIN-4-YL-7H-PYRROLO[2,3-D]PYRIMIDIN-4-YL)PHENYL]-3-(TRIFLUOROMETHYL)BENZAMIDE (COMPOUND NO. 101)

### Step 1: Ethyl 2-amino-5-pyridin-4-yl-1H-pyrrole-3-carboxylate

To an ethyl alcohol (10 mL) was added sodium metal (0.23 g, 10.0 mmol), and the reaction mixture stirred for 20 minutes until complete solution was obtained. This sodium ethoxide solution was added in another flask to a cold (0-5° C.) suspension of amidinoacetic acid ethyl ester (4.0 g, 24.0 mmol) (prepared as described by: T. Kobayashi, Chem. Pharm. Bull. 43(5), 788-796, 1995) in ethyl alcohol (5 mL), and the resulting mixture was stirred at 5° C. for 20 minutes. The ethyl bromopyruvate (0.63 mL, 5.0 mmol) was added and stirred at 5° C. for 30 minutes and then stirred at room temperature overnight. The solvent was evaporated to dryness and the residual oil was washed with ethyl acetate and methylene chloride. The ethylacetate and methylene chloride washes were combined and evaporated to yield an oil. The oil was purified by silica gel flash chromatography to yield 1.04 g (37%) of as a yellow solid, mp. 193°-195° C. MS: m/z 232.2 [M+H].

### Step 2: 6-pyridin-4-yl-7H-pyrrolo[2,3-d]pyrimidin-4-ol

A mixture of ethyl 2-amino-5-pyridin-4-yl-1H-pyrrole-3-carboxylate (1.0 g, 4.32 mmol), formamide (10.4 mL), N,N-dimethyl formamide (5.2 mL) and formic acid (2.6 mL) was stirred and heated at 150° C. for 2 days. The reaction mixture was cooled and the solid collected by filtration, washed with water and dried to yield 0.5 g (55%) of title compound as a tan solid, mp. >260° C. MS: m/z213.1 [M+H].

### Step 3: 4-chloro-6-pyridin-4-yl-7H-pyrrolo[2,3-d]pyrimidine

A mixture of 6-pyridin-4-yl-7H-pyrrolo[2,3-d]pyrimidin-4-ol (0.47 g, 2.2 mmol), 2 drops of N,N-dimethyl formamide and phosphorus oxychloride (8 mL) was heated at 120° C. for 20 hours. After cooling the mixture, excess phosphorus oxychloride was evaporated, followed by addition of toluene and subsequent evaporation to further remove phosphorus oxychloride. Addition and evaporation of toluene was repeated, and the dark residue was cooled with an ice bath and stirred with cold saturated solution of sodium bicarbonate. The solid was collected by filtration, washed with water and dried to yield 0.56 g of title compound as a dark solid that was used without purification in the next step. MS: m/z 231.1 [M+H].

### Step 4: N-[3-(6-pyridin-4-yl-7H-pyrrolo[2,3-d]pyrimidin-4-yl)phenyl]-3-(trifluoromethyl)benzamide

To a solution of 4-chloro-6-pyridin-4-yl-7H-pyrrolo[2,3-d]pyrimidine (0.12 g, 0.56 mmol) in DME (9 mL)was added (3-{[3-(trifluoromethyl)benzoyl]amino}phenyl)boronic acid (0.38 g, 1.2 mmol), tetrakis(triphenylphosphine)palladium(0) (45 mg, 0.039 mmol) and saturated solution of sodium bicarbonate (3 mL)). After microwaving at 100° C. for 600 seconds, the solution was concentrated and the residue was purified by silica gel flash chromatography (Methanol/CH₂Cl₂) to yield 0.15 g (58%) of title compound as a white solid. Mp>280° C. MS: m/z 460.3 [M+H].

### Example 19

### N-{3-[6-PYRIDIN-4-YL-7-(2-PYRROLIDIN-1-YLETHYL)-7H-PYRROLO[2,3-D]PYRIMIDIN-4-YL]PHENYL}-3-(TRIFLUOROMETHYL)BENZAMIDE (COMPOUND NO. 108)

A mixture of N-[3-(6-pyridin-4-yl-7H-pyrrolo[2,3-d]pyrimidin-4-yl)phenyl]-3-(trifluoromethyl)benzamide (80 mg, 0.17 mmol), sodium hydride (60% in oil) (15 mg, 0.38 mmol) and N-(2-chloroethyl) pyrrolidine hydrochloride (30 mg, 0.17 mmol) in DMF (0.8 mL) was heated at 80° C. for 7 hours. As the reaction was not complete, the mixture was cooled and to it was added 15 mg of N-(2-chloroethyl) pyrrolidine hydrochloride, 10 mg of sodium hydride (60% in oil) and the resulting mixture was heated at 80° C. overnight. After cooling, the mixture was stirred with a saturated solution of sodium bicarbonate. The solid was collected by filtration, washed with water and dried. The crude product was purified by silica gel flash chromatography (Methanol/CH₂Cl₂) to yield 16 mg (17%) of N-{3-[6-pyridin-4-yl-7-(2-pyrrolidin-1-ylethyl)-7H-pyrrolo[2,3-d]pyrimidin-4-yl]phenyl}-3-(trifluoromethyl)benzamide as a yellow foam. MS: m/z 557.2 [M+H].

N-(3-{7-[3-(dimethylamino)propyl]-6-pyridin-4-yl-7H-pyrrolo[2,3-d]pyrimidin-4-yl}phenyl)-3-(trifluoromethyl)benzamide 109 was prepared following the procedure described for example 19 using the corresponding starting materials. MS: m/z 545.2 [M+H].

### Example 20

### N-(3-(7-(4-METHOXYPHENYL)-7H-PYRROLO[2,3-D]PYRIMIDIN-4-YL)PHENYL)-3-(TRIFLUOROMETHYL)BENZAMIDE (COMPOUND NO. 103)

A mixture of N-(3-(7H-pyrrolo[2,3-d]pyrimidin-4-yl)phenyl)-3-(trifluoromethyl)-benzamide (0.15 g, 0.4 mmol), 4-methoxyphenylboronic acid (40 mg, 0.26 mmol), copper (II) acetate (47 mg, 0.26 mmol), pyridine (21 μL, 0.26 mmol), and molecular sieves in THF (2 mL) were stirred for 4 days at room temperature. The reaction mixture was filtered, concentrated and purified by reverse phase chromatography to give N-(3-(7-(4-methoxyphenyl)-7H-pyrrolo[2,3-d]pyrimidin-4-yl)phenyl)-3-(trifluoromethyl)benzamide (63 mg, 37% yield). MS: m/z 489.4 [M+H].

Compound Numbers 105 and 107 were prepared following the procedure described for Example 20 using the corresponding starting materials.

| Compound | | |
|---|---|---|
| No. | Compound Name | MS, m/z |
| 105 | N-{3-[7-(4-formylphenyl)-7H-pyrrolo[2,3- | 487.1 [M + H] |
| | d]pyrimidin-4-yl]phenyl}-3- | |
| | (trifluoromethyl)benzamide | |
| 107 | N-(3-{7-[4-(dimethylamino)phenyl]-7H- | 502.3 [M + H] |
| | pyrrolo[2,3-d]pyrimidin-4-yl}phenyl)- | |
| | 3-(trifluoromethyl)benzamide | |

### Example 21

### N-(3-(7-(4-((DIMETHYLAMINO)METHYL)PHENYL)-7H-PYRROLO[2,3-D]PYRIMIDIN-4-YL)PHENYL)-3-(TRIFLUOROMETHYL)BENZAMIDE (COMPOUND NO. 111)

A mixture of N-(3-(7H-pyrrolo[2,3-d]pyrimidin-4-yl)phenyl)-3-(trifluoromethyl)-benzamide (0.12 g, 0.31 mmol), 5-iodopyrimidin-2-amine (67 mg, 0.0.31 mmol), copper (I) iodide (24 mg, 0.13 mmol), potassium phosphate (140 mg, 0.66 mmol), and N,N-dimethylcyclohexane-1,2-diamine (31 mg, 0.22 mmol) in DMF (0.5 mL) were stirred for 24 hours at 100° C. The reaction mixture was filtered and purified by reverse phase chromatography to give N-(3-(7-(4-((dimethylamino)methyl)phenyl)-7H-pyrrolo[2,3-d]pyrimidin-4-yl)phenyl)-3-(trifluoromethyl)benzamide (63 mg, 32% yield). MS: m/z 516.4 [M+H].

Compound Numbers 100 and 112 were prepared following the procedure described for example 21 using the corresponding starting materials.

| Compound | | |
|---|---|---|
| No. | Compound Name | MS, m/z |
| 100 | N-(3-{7-[4-(2-methyl-1H-imidazol-1- | 553.2 [M + H] |
| | yl)benzyl]-7H-pyrrolo[2,3-d]pyrimidin-4- | |
| | yl}phenyl)-3-(trifluoromethyl)benzamide | |
| 112 | N-(3-{7-[4-(piperazin-1-ylmethyl)phenyl]- | 557.4 [M + H] |
| | 7H-pyrrolo[2,3-d]pyrimidin-4- | |
| | yl}phenyl)-3-(trifluoromethyl)benzamide | |

While the present invention has been described with respect to particular disclosed embodiments, it should be understood that numerous other embodiments are within the scope of the present invention. Accordingly, the invention is not to be considered as being limited by the foregoing description and drawings, but is only limited by the scope of the appended claims.

What is claimed is:

1. A compound of formula Ia 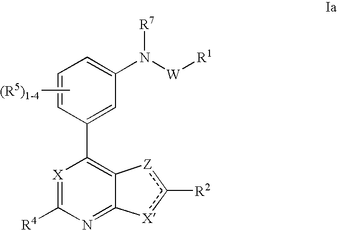
and pharmaceutically acceptable salts thereof;
wherein R¹ is selected from the group consisting of phenyl, heterocyclic ring and heteroaryl ring containing 1 to 3 ring heteroatoms selected from nitrogen, oxygen, and sulfur, wherein said phenyl, heterocyclic, and heteroaryl ring are each optionally substituted with from one to four substituents independently selected from the group consisting of -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, and YR¹⁰;
R² and R³ are independently selected from the group consisting of —H, -J, —C(O)OR⁷, —C(O)NR⁷R⁷, —NR⁶C(O)R⁷, —CN, heterocyclic ring and heteroaryl ring having 5 to 7 ring atoms and containing 1 to 3 ring heteroatoms selected from nitrogen, oxygen, and sulfur, and C₆-C₁₄ aryl ring, wherein —R⁷, heterocyclic, heteroaryl and aryl rings are optionally substituted with from one to four substituents independently selected from the group consisting of -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, ——N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, and —YR¹⁰;
R⁴ is selected from the group consisting of —H, C₁-C₈ alkyl, C₂-C₆ alkenyl, and C₂-C₆ alkynyl;
R⁵, at each occurrence, is from one to four substituents independently selected from the group consisting of —H, -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, and YR¹⁰; —R⁶, at each occurrence, is selected from the group consisting of —H, —C(O)OR⁷, —C(O)NR⁷R⁷, C₃-C₁₀ carbocyclic ring, heterocyclic ring and heteroaryl ring having 5 to 7 ring atoms and containing 1 to 3 ring heteroatoms selected from nitrogen, oxygen, and sulfur, and C₆-C₁₄ aryl ring, wherein said R⁷, carbocyclic ring, heterocyclic ring, heteroaryl ring, and aryl ring are each optionally substituted with from one to four substituents independently selected from the group consisting of -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷, —OC(O)OR⁷, —OC(O)NR⁷R⁷, NR⁷C(O)R⁷, —NR⁷C(O)OR⁷, —NR⁷C(O)NR⁷R⁷, —R⁸OR⁷, —R⁸NR⁷R⁷, —R⁸S(O)ₘR⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, —R⁸NR⁷C(O)NR⁷R⁷, and —R¹⁰;
R⁷, at each occurrence, is selected from the group consisting of —H, C₁-C₈ alkyl, C₂-C₆ alkenyl, and C₂-C₆ alkynyl;
R⁸ is a divalent group selected from the group consisting of C₁-C₆ alkyl, C₂-C₆ alkenyl, and C₂-C₆ alkynyl;
R⁹ is a divalent C₂-C₆ alkyl group;
R¹⁰, at each occurrence, is selected from the group consisting of a C₃-C₁₀ carbocyclic ring; a heterocyclic ring and a heteroaryl ring containing 1 to 3 ring heteroatoms selected from nitrogen, oxygen, and sulfur; and C₆-C₁₄ aryl ring; wherein the carbocyclic ring, heterocyclic ring, heteroaryl ring, and aryl ring are optionally substituted with from one to four substituents independently selected from the group consisting of —H, -J, —NO₂, —CN, —N₃, —CHO, —CF₃, —OCF₃, —R⁷, —OR⁷, —S(O)ₘR⁷, —NR⁷R⁷, —NR⁷S(O)ₘR⁷, —OR⁹OR⁷, —OR⁹NR⁷R⁷, —N(R⁷)R⁹OR⁷, —N(R⁷)R⁹NR⁷R⁷, —NR⁷C(O)R⁷, —C(O)R⁷, —C(O)OR⁷, —C(O)NR⁷R⁷, —OC(O)R⁷—, —OC(O)OR⁷, —OC(O)NR⁷R⁷, —NR⁷C(O)R⁷, —NR⁷C(O)OR⁷, —NR⁷C(O)N R⁷R⁷, —R⁸OR⁷, R⁸NR⁷R⁷, —R⁸S(O)ₘR⁷, —R⁸C(O)R⁷, —R⁸C(O)OR⁷, —R⁸C(O)NR⁷R⁷, —R⁸C(O)R⁷, —R⁸C(O)OR⁷, —R⁸C(O)NR⁷R⁷, —R⁸OC(O)R⁷, —R⁸OC(O)OR⁷, —R⁸OC(O)NR⁷R⁷, —R⁸NR⁷C(O)R⁷, —R⁸NR⁷C(O)OR⁷, and —R⁸NR⁷C(O)NR⁷R⁷;
J is selected from fluoro, chloro, bromo, and iodo;
m is an integer from 0 to 2;
W is —C(O)— or —C(O)NR⁷—;
X is N, C—CN or C—C(O)NH₂;
X′ is —S—, —N(R⁶)—, or ═C(R³)—;
Z is —S— or ═C(R³)—, with the proviso wherein only one of X′ and Z is ═C(R³)—;
Y is selected from a bond, —NH—, —O—, —NR⁷—, and R⁸; and [symbol]
represents a single bond or a double bond.

2. The compound according to claim 1 wherein X is N; X′ is —N(R⁶)—; and Z is ═C(R³)—.

3. The compound according to claim 2 where R⁶ is selected from the group consisting of methylsulfonyl, phenylsulfonyl, tosyl, pyridin-4-yl, 4-(piperazin-1-ylmethyl)phenyl, 2-aminopyrimidin-5-yl, N-methylpicolinamide, 4-(dimethylamino)methylphenyl, 3-dimethylaminopropyl, and (2-(pyrrolidin-1-yl)ethyl).

4. The compound according to claim 1 wherein X is N; X′ is —S—; and Z is ═C(R³)—.

5. The compound according to claim 1 wherein X is N; X′ is ═C(R³)—; and Z is —S—.

6. The compound according to any of claims 1-5 wherein R¹ is selected from the group consisting of 3-trifluoromethylphenyl, 4-fluoro-3-trifluoromethylphenyl, 4-methyl-3-trifluoromethylphenyl, 4-chloro-3-trifluoromethylphenyl, 4-methoxy-3-trifluoromethylphenyl, 3,4-dimethylphenyl 4-methylphenyl, 3-methylphenyl, 2-methylphenyl, 4-bromophenyl, 3-bromophenyl, 2-bromophenyl, 4-fluorophenyl, 3-fluorophenyl, 2-fluorophenyl, 4-chlorophenyl, 3-chlorophenyl, 2-chlorophenyl and 3,4-dichlorophenyl.

7. The compound according to any of claims 1-5 wherein R² is selected from the group consisting of 4-methylphenyl, 3-methylphenyl, 2-methylphenyl, 4-aminophenyl, 4-acetamidophenyl, 4-chlorophenyl, 3-chlorophenyl, 2-chlorophenyl, 4-hydroxypiperidin-4-yl, N-methyl-5-picolinamido, N,N-dimethyl-4-carbamoylphenyl, N,N-dimethyl-3-carbamoylphenyl, 4-acetylphenyl, and 3-acetylphenyl.

8. A method for making a compound of formula Ia according to claim 1 and pharmaceutically acceptable salts thereof, comprising
(a) reacting, in the presence of a palladium catalyst, a heterocyclic compound of formula II 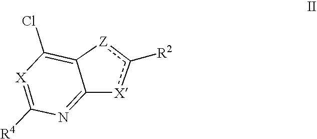
with a boronic acid of formula III, 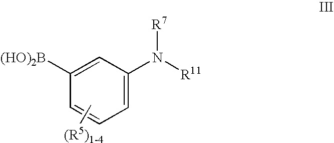
to obtain a compound of formula If, 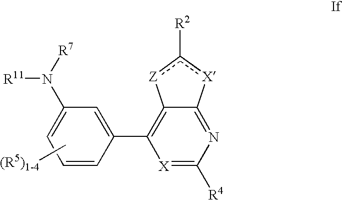
wherein R¹¹ is —H or —W—R¹;
and, step (b) when R¹¹ is H reacting the compound If obtained with an electrophile containing the —W—R¹ radical.

9. The method according to claim 8 wherein the electrophile containing the —W—R¹ radical is selected from the group of acid chlorides, isocyanates, anhydrides, 1-hydroxybenzotriazole esters, and mixed carboxylic phosphoric acid anhydrides.

10. The method according to claim 8 wherein the heterocyclic compound of formula II is prepared by a process comprising the steps of
(a) reacting a compound of formula VI wherein X′ is —S— or —N(R⁶)—, 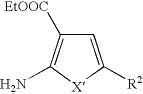
with a formamidine compound of formula VII 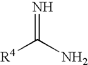
to obtain a compound of formula VIII 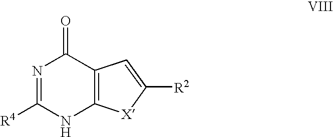
and, (b) reacting compound VIII with a chlorinating agent.

11. A pharmaceutical composition comprising a compound according to any of claims 1-7 and a pharmaceutically acceptable carrier.

12. A pharmaceutical composition comprising a compound according to any of claims 1-7 in combination with other kinase-inhibiting pharmaceutical compositions or chemotherapeutic agents, and a pharmaceutically acceptable carrier.

13. A method of inhibiting kinase activity in a mammal comprising administering to a mammal a kinase-inhibiting amount of a compound according to any of claims 1-7.

14. The method of claim 13, wherein the mammal is a human.

15. A method of treating a kinase-dependent condition comprising administering to a subject a kinase-inhibiting amount of a pharmaceutical composition according to any of claims 1-7.

16. A method of treating a B-Raf kinase-dependent condition comprising inflammation or cancer, by administering to a patient a compound of any of claims 1-7.

17. The method of claim 16, wherein the cancer is cancer is selected from the group consisting of breast, kidney, bladder, mouth, larynx, esophagus, stomach, colon, ovary, lung, pancreas, skin, liver, prostate and brain cancer.

18. A method of treating cancer, by administering to a patient a compound of any of claims 1-7.

19. The method of claim 18, wherein the cancer is cancer is selected from the group consisting of breast, kidney, bladder, mouth, larynx, esophagus, stomach, colon, ovary, lung, pancreas, skin, liver, prostate and brain cancer.
